SEQUENCE LISTING (4 sequences)

SEQ ID NO: 1 - protein, 1229 aa, Homo sapiens
  MISC_FEATURE (1)..(10): His-tag
MHHHHHHEFPMATLEKLMKAFESLKSFQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVALASCDLTSSATDGDEEAILSHSSSQVSAVPSDPAMDLNAGTQASSPISDSSQTTTEGPDSAVTPSDSSEIVLDGTANQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRVEGYPYDVPDYA

SEQ ID NO: 2 - protein, 301 aa, Homo sapiens
  MISC_FEATURE (270)..(301): DDK-tag
MAFYKREMFDPAEKYKMDHRRRGIALIFNHERFFWHLTLPERRGTCADRDNLTRRFSDLGFEVKCFNDLKAEELLLKIHEVSTVSHADADCFVCVFLSHGEGNHIYAYDAKIEIQTLTGLFKGDKCHSLVGKPKIFIIQACRGNQHDVPVIPLDVVDNQTEKLDTNITEVDAASVYTLPAGADFLMCYSVAEGYYSHRETVNGSWYIQDLCEMLGKYGSSLEFTELLTLVNRKVSQRRVDFCKDPSAIGKKQVPCFASMLTKKLHFFPKSNTRTPLEQKLISEEDLAAMISWITRDDDDKV

SEQ ID NO: 3 - protein, 1229 aa, Homo sapiens
  MISC_FEATURE (1)..(10): His-tag
MHHHHHHEFPMATLEKLMKAFESLKSFQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRVEGYPYDVPDYA

SEQ ID NO: 4 - protein, 323 aa, Homo sapiens
  MISC_FEATURE (292)..(323): DDK-tag
MSSASGLRRGHPAGGEENMTETDAFYKREMFDPAEKYKMDHRRRGIALIFNHERFFWHLTLPERRGTCADRDNLTRRFSDLGFEVKCFNDLKAEELLLKIHEVSTVSHADADCFVCVFLSHGEGNHIYAYDAKIEIQTLTGLFKGDKCHSLVGKPKIFIIQACRGNQHDVPVIPLDVVDNQTEKLDTNITEVDAASVYTLPAGADFLMCYSVAEGYYSHRETVNGSWYIQDLCEMLGKYGSSLEFTELLTLVNRKVSQRRVDFCKDPSAIGKKQVPCFASMLTKKLHFFPKSNTRTPLEQKLISEEDLAAMISWITRDDDDKV

(19) United States
(12) Patent Application Publication
(10) Pub. No.: US 2019/0192524 A1
(43) Pub. Date: Jun. 27, 2019

(54) Modulators of Caspase-6

(71) Applicants: The University of British Columbia, Vancouver (CA); University of Hamburg, Hamburg (DE)

(72) Inventors: Mehdi Mike Khankischpur, Hamburg (DE); Olga Petina, Dueren (DE); Detief Gettken, Lueneburg (DE); Dabmar Ehmhoefer, Vancouver (CA); Michael Hayden, Vancouver (CA)

(21) Appl. No.: 15/118,452

(22) PCT Filed: Feb. 14, 2015
(86) PCT No.: PCT/IB2015/000144

§ 371 Date: Aug. 11, 2016

Related U.S. Application Data
(60) Provisional application No. 62/033,401, filed on Aug. 5, 2014.
(60) Provisional application No. 61/939,922, filed on Feb. 14, 2014.

(51) Int. Cl. A61K 31/535; A61P 25/28; A61K 31/55; A61K 31/4375; A61K 31/27; A61K 31/445; A61K 31/13; A61K 31/135; A61K 31/198

(52) U.S. Cl. CPC A61K 31/535; A61P 25/28; A61K 31/55; A61K 31/4375; A61K 31/198; A61K 31/445; A61K 31/13; A61K 31/135; A61K 31/27

(57) ABSTRACT

Modulators of caspase-6 activity are provided for use in the treatment of neurodegenerative diseases.

### CROSS-REFERENCE TO RELATED APPLICATIONS

The application is a national filing of PCT/IB2015/000144, filed Feb. 14, 2015, and claims benefit of priority to U.S. Provisional Application No. 62/033,401 filed Aug. 5, 2014 and U.S. Provisional Application No. 61/939,922, filed Feb. 14, 2014, the entire disclosures of each of which is incorporated herein in its entirety by reference.

### FIELD OF THE INVENTION

The present invention relates to the field of therapies for neurodegenerative diseases. More particularly, compounds and methods of using compound that modulate caspase-6 activity.

### BACKGROUND

Huntington disease (HD) is a progressive neurodegenerative disorder with an autosomal dominant inheritance pattern characterized by chorea, cognitive decline and behavioural changes that usually manifest in mid-adulthood. HD is caused by a mutant HD gene (mhtt) that contains tandem CAG repeats coding for a polyglutamine region. The number of CAG repeats correlates inversely with age of onset of the disease. Neurodegeneration occurs initially and most severely in the medium-spiny neurons of the striatum, and later in the cortex. There is strong evidence to suggest that apoptosis plays a role in the neurodegeneration observed in HD. Proteolytic cleavage of mhtt is a crucial factor in the pathogenesis of HD.

Htt is proteolytically cleaved by caspases, releasing an amino terminal fragment containing the glutamine tract (Wellington et al. 2000 J. Biol Chem 275(26):19831-8). In particular, the expression of mhtt fragments containing an expanded polyglutamine repeat is toxic in vitro and in vivo, and accumulation of N-terminal truncated products of mhtt is observed in human and mouse HD brain. Caspase-6 (C6) is a cysteine-aspartic acid protease that, when activated, cleaves htt and mhtt. Studies in mice have shown that that the expression of mhtt that is resistant to C6 cleavage (C6R) significantly reduces htt toxicity and leads to a dramatic improvement of the phenotype, whereas mice expressing mhtt resistant to cleavage by caspases-3 (C3) and -2 (C2) were not protected (Graham et al. 2006. Cell 125(6):1179-91). Dramatically, mice expressing C6R mhtt do not demonstrate increased C6 activation. The absence of C6 activation in C6R mice suggests that the 586aa htt fragment may be part of a forward amplification cycle of C6 activation in HD (Graham, R. K., et al., J Neurosci, 2010. 30(45): p. 15019-29). These data suggest that mhtt fragments generated by C6 cleavage may be required to initiate a toxic cycle that leads to neuronal dysfunction and the neuropathological abnormalities in HD (Graham et al. Trends Neurosci, 2011. 34(12): p. 646-56).

Caspase-6 (C6) mRNA is increased in early grade (0-2) human HD caudate and motor cortex compared to control tissue (Hodges et al. Hum Mol Genet, 2006. 15(6): p. 965-77), and active C6 is also present in presymptomatic and early-grade human and murine HD brain, where as other executioner caspases such as C3 are not activated at these stages. Intriguingly, active C6 levels correlate with CAG size in human HD brain and inversely correlate with age of onset. This evidence implies a relationship between C6 and htt, with the size of the CAG tract influencing levels of C6 activation and thereby contributing to the disease process.

Similarly to htt, the amyloid precursor protein (APP), fragments of which accumulate and are thought to be pathogenic in Alzheimer's disease (AD), is also cleaved by caspases during apoptosis (Ayala-Grosso, C., et al., Brain Pathol, 2002. 12(4): p. 430-41). Two characterized sites in the amino terminus of Aβ include V(K or N)(M or L)D653 and the VEVD664, both of which correspond to C6 recognition motifs (Gervais et al. Cell 1999. 97(3)L 395-406). Cleavage of APP at the D664 site is detected early in human AD brain tissue and is a necessary requirement for nuclear p21-activated kinase signaling (Nguyen et al, J. Neurochem, 2008. 22(3):703-12) and the deficits in synaptic transmission, synaptic plasticity and learning observed in the pathophysiology of AD (Saganich et al, J. Neuroscience, 2006. 26(52): 13428-36). Importantly, and providing proof of principle for the importance of cleavage at this site in the etiology of AD, inhibiting cleavage of APP at aa664 provides some rescue from behavioural deficits and neuropathology in an AD mouse model (Zhang et al., Behav Brain Res, 2010. 206(2):202-207; Galvan et al, PNAS, 2006. 103(18):7130-5).

AD patient brain tissue also exhibits a significant increase in C6 mRNA compared to controls and active C6 (Pompl et al., Arch Neurol, 2003. 60(3):369-376). C6-cleaved tau and C6 cleaved α-tubulin are highly abundant in neuropil threads, neurofibrillary tangles and neuritic plaques of AD brain (Guo et al., Am J Pathol, 2004. 165(2): 523-531; Klaiman et al., Cell Proteomics, 2008. 7(8):1541-55). Interestingly, C6-cleaved tau levels negatively correlate with global cognitive scores, declarative and semantic memories in the brains of aged noncognitively impaired individuals suggesting that the activity of C6 precedes the clinical and pathological diagnosis of AD (Albrecht et al. Am J Pathol, 2007, 170(4): p. 1200-9; LeBlanc et al. Cell Death and Differentiation, 2014. 21:696-706).

The role of C6 in neuronal dysfunction and specifically in axonal degeneration is supported by other studies, demonstrating that C6 is activated in degenerating neurons and axons post trophic factor withdrawal (Park et al. Nat Neurosci, 2010. 13(5): p. 559-66). Furthermore, C6 is activated and required for the neurodegeneration following ischemic insult (Akpan et al., J Neurosci, 2011, 31(24): p. 8894-904). Taken together these studies support inhibitors of C6 as therapeutic strategies for neurological diseases.

Most caspase inhibitors used to validate the role of caspases in disease processes are peptide-based and mimic the cleavage site present in caspase substrates. Non-peptide inhibitors have been described for C6, when tested in an in vitro model of HD, the pan-caspase inhibitors described by Levya and coworkers showed protection from mhtt toxicity (Leyva et al Chem Biol, 2010. 17(11): p. 1189-200). More recently, Murray and colleagues have described small molecules that bind procaspase 6 at an allosteric site (Murray et al Chem Med Chem, 2014. 9(1): 73-77).

### SUMMARY

In certain aspects, the present disclosure can be described as a pharmaceutical composition for the treatment of a neuro generative disease, wherein the composition includes a caspase-6 inhibitor and one or more pharmaceutical carriers or excipients. A caspase-6 inhibitor can be a small molecule, such as an arylpropynamide derivative and can have one of the following structures I or II or a pharmaceutically acceptable salt, stereoscopic isomer, derivative or prodrug thereof:

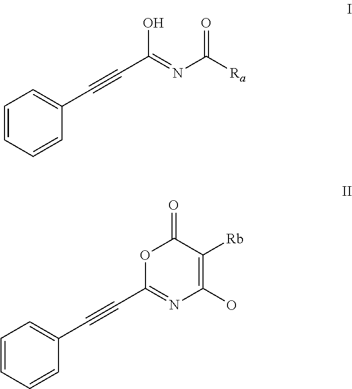

Wherein Ra and Rb are independently linear or branched C₁ to C₆ alkyl, aryl, or alkenyl, C₁ to C₉ alkylaryl, C₁ to C₉ substituted alkylaryl, or C₁ to C₉ alkylheteroaryl and

wherein the phenyl is substituted with 0, 1 or 2 halogens and;

further wherein the halogens are chloride, fluoride or bromide.

The present disclosure can also be described in certain embodiments as a method of treating a neurological disease, the method including administering to a subject in need of such treatment an effective dose of a pharmaceutical composition that includes a therapeutically effective amount of a compound having the structure of either structure I or II as described herein, or a pharmaceutically acceptable salt, stereoscopic isomer, derivative or prodrug thereof; wherein the disease is Huntington's disease, Alzheimer's disease, dementia, mild-cognitive impairment, or memory loss. The composition can be administered either alone or as an adjunct or combination therapy with administration of one or more drugs or agents useful in the treatment of neurological disease. The combination of drugs can be administered in the same formulation or separately and can be administered simultaneously or sequentially. Exemplary drugs useful for the treatment of neurological disease are L-DOPA, rasagiline, memantine hydrochloride, donepezil hydrochloride, rivastigmine, galantamine, tetrabenzine.

The methods of treatment described herein can be commenced after the onset of symptoms in a subject or treatment can begin prior to the presentation of symptoms of neurological disease in a subject susceptible to or suspected of being susceptible to neurological disease. A subject susceptible to or suspected of being susceptible to neurological disease can be identified by the presence of expression of a mutant htt gene in the subject, by overexpression of caspase-6 mRNA in neural cells of the subject relative to expression of caspase-6 mRNA in a healthy subject or by the presence of axonal degeneration in a subject as evidenced by white mass loss on magnetic resonance imaging.

It is understood that term “subject” as used herein can designate a human subject or a veterinary subject.

In certain embodiments the disclosed compositions can include a pharmaceutical composition for the treatment or amelioration of a neurological disease wherein the composition comprises a therapeutically effective amount of an active agent with a structure as shown in FIG. 3, or pharmaceutically acceptable salt, stereoscopic isomer, derivative or prodrug thereof; in which the active agent has the structure designated as one of PG-3a-h or PG-3.

The disclosure can further be defined as compositions and methods for the treatment of neurodegenerative diseases, such as Huntington's disease, Alzheimer's disease, dementia, mild-cognitive impairment, or memory loss, for example. For treatment of neurodegeneration, the disclosed compositions can be formulated to be administered orally, topically or parenterally. While systemic administration such as oral administration or parenteral routes such as injection or infusion into the general circulation is possible, the use of such routes for actives targeted to the brain and central nervous system may require undesirably high serum levels in order to achieve therapeutic levels in the CNS. As such the disclosed compositions can also be formulated for direct introduction to the brain through intracerebroventricular or intraparenchymal injections, or for intranasal delivery via the olfactory bulb or trigeminal nerve, for example.

For injection, a formulation of a therapeutic amount of the disclosed caspase-6 inhibitors or their pharmaceutically acceptable salts are prepared for subcutaneous, intravenous, or intramuscular injection, or a formulation is prepared for direct introduction into the brain or CSF by intracerebroventricular, intraparenchymal or intrathecal injection. In addition to the active ingredient, these pharmaceutical compositions can contain a buffer, to maintain the pH in the range from 3.5 to 7 and also a salt such as sodium chloride, and can also contain mannitol or sorbitol for adjusting the isotonic pressure. In certain embodiments, DMSO or another organic solvent can be added.

When the composition is formulated for intravenous, intraperitoneal or subcutaneous administration, by infusion or injection, for example, solutions of the active compound or its salts can be prepared in water and optionally mixed with a surfactant. Dispersions can also be prepared in glycerol, liquid polyethylene glycols, triacetin, and mixtures thereof and in oils. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. Such compositions can also include isotonic agents like sugars, buffers or sodium chloride and optionally absorption delay agents such as aluminum monostearate, cellulose ethers, and gelatin.

It is another aspect of the formulations that they may also include lipids to facilitate penetration of the compositions into the tissues of the CNS. Exemplary lipids useful for the disclosed compositions include, but are not limited to phosphatidyl choline and cholesterol. The disclosed compositions can also be administered as oil in water emulsions in which the oils are selected from a synthetic oil or a plant oil, including for example, olive oil, soybean oil, cottonseed oil, soybean oil, sesame oil, sunflower oil, safflower oil, avocado oil, peanut oil, walnut oil, almond oil or hazelnut oil.

The disclosed compositions can also be prepared in certain embodiments as a liposome preparation, wherein the liposome is a large unilamellar vesicle (LUV), a multilammelar vesicle (MLV), or a small unilamellar vesicle (SUV). The LUV has a particle system ranging from about 200 to about 1000 nm. The MLV has a particle system ranging from about 400 to about 3500 nm. The SUV has a particle system ranging from about 20 to about 50 nm.

Examples of lipids for forming a liposome include phospholipids, cholesterols, or nitrogen lipids. Phospholipids can be selected from naturally occurring phospholipids, such as phosphatidylcholine, phosphatidylserine, phosphatidylglycerol, phosphatidylinositol, phosphatidylethanolamine, phosphatidic acid, cardiolipin, sphingomyelin, egg yolk lecithin, soybean lecithin, lysolecithin, for example or the corresponding phospholipids hydrogenated or synthetic phospholipids, such as dicetylphosphate, distearoylphosphatidylcholine, dipalmitoylphosphatidylcholine, dipalmitoylphosphatidylethanolamine, dipalmitoylphosphatidylserine, eleostearoylphosphatidylcholine, eleostearoylphosphatidylethanolamine, and eleostearoylphosphatidylserine.

It is well known that delivery of pharmaceutical agents to the brain and central nervous system is challenging because of the blood brain barrier (BBB) which inhibits or prevents delivery of small and large molecules to the brain from the circulating blood. The disclosed formulations, therefore, can be formulated for inhalation, or intranasal delivery to the brain. In order for a therapeutic to have adequate absorption and bioavailability in the CNS, a solubility agent can be required. In certain embodiments the active ingredient is encapsulated in a carrier, such as cyclodextrins, microemulsions, or nanoparticles for intranasal delivery to the CNS. Cyclodextrin inclusion complexes containing a hydrophobic cavity surrounded by a hydrophilic shell can improve the solubility of poorly water-soluble drugs, thus enhancing brain uptake after intranasal administration. Polymeric nanoparticles, with a hydrophobic core of polylactic acid (PLA) and a hydrophilic shell of methoxy-poly(ethylene glycol) (MPEG), are also contemplated for use in the disclosed compositions for improving solubility and intranasal drug targeting to the CNS.

Efficient delivery to the CNS following intranasal administration is also dependent on membrane permeability, as improving membrane permeability can enhance transport to the CNS along olfactory and trigeminal nerves. Additionally, the compositions may be used in conjunction with permeation enhancers, such as surfactants, bile salts, lipids, cyclodextrins, polymers, and tight junction modifiers.

In certain embodiments the disclosed compositions can be formulated for oral administration. Such a formulation can be in the form of a tablet, capsule, or beads, or a liquid, gel, or syrup, for example. The form and strength of the oral delivery composition and is determined by absorbance characteristics, tolerable or safe serum drug concentrations and ability to deliver an effective amount of drug to the CNS.

Solid oral compositions can be prepared as tablets, powder or beads and can be formulated for immediate release, sustained or controlled release, delayed release or combinations thereof, in order to achieve release in a chosen portion of the gastrointestinal system, to maintain an effective dose over a longer time period, or to deliver a selected dose range at a particular time, for example. Sustained, controlled and delayed release formulations often entail the use of coating layers over an active pharmaceutical ingredient (API) core or layer.

Orally administered tablets, troches, pills, capsules, etc. can also contain binders, excipients, disintegrating agents, lubricants, flavoring or sweetening agents, buffers, salts, sugars, and coatings of polymers, both water soluble and water insoluble, film formers and plasticizers.

A liquid formulation can contain the drug in a solution, emulsion, suspension, or colloidal suspension, for example, in the appropriate solvents and excipients. A syrup or elixir can contain additional thickeners or viscosity agents as necessary.

A composition for topical application or infusion can be formulated as an aqueous solution, lotion, jelly or an oily solution or suspension. A composition in the form of an aqueous solution is obtained by dissolving a caspase-6 inhibitor in solvent and a buffer solution and optionally a polymeric binder. An oily formulation for topical application is obtained by suspending the caspase-6 inhibitor in an oil, optionally with the addition of a swelling agent such as aluminum stearate and/or a surfactant. An absorbance enhancer such as DMSO can also be added to a topical composition for transdermal administration.

Unless otherwise specified or defined within the context of use, all terms herein are meant to convey their normal meaning as used in the respective arts and as discussed below.

As used herein, “pharmaceutically acceptable carrier” includes any and all non-active or inert ingredients, including but not limited to solvents, dispersion media, coatings, antibacterial and antifungal agents, isotonic and absorption influencing agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

As used herein, the term “pharmaceutically acceptable salt” refers to non-toxic pharmaceutically acceptable salts as described (Ref. International J. Pharm., 1986, 33, 201-217; J. Pharm. Sci., 1997 (January), 86, 1, 1). Other salts well known to those in the art may, however, be useful in the preparation of compositions of the disclosure including, but not limited to, hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. Representative organic or inorganic bases include, but are not limited to, basic or cationic salts such as benzathine, chloroprocaine, choline, diethanolamine, ethylenediamine, meglumine, procaine, aluminum, calcium, lithium, magnesium, potassium, sodium and zinc.

The compositions and active agents of this disclosure are administered in an “effective amount,” “effective dose,” or “therapeutically effective amount or dose.” By an “effective” amount or a “therapeutically effective amount” or dose of a drug or pharmacologically active agent is meant a nontoxic but sufficient amount of the drug or agent to provide the desired effect. In the current disclosure, an “effective amount” is the amount of that composition or active agent that is effective to improve, ameliorate or prevent one or more symptoms of the condition being treated. The amount that is “effective” will vary from subject to subject, depending on the age, weight and general condition of the individual, or the particular active agent. A therapeutic or effective dose or amount is determined by a physician and is often based on empirical data obtained by administering increasing doses until the best balance of benefit vs. side effects is reached.

The term “parenteral” as used herein includes all non-oral delivery techniques including transdermal administration, subcutaneous injection, intravenous, intramuscular or intrasternal injection, intrathecal injection (directly into the CNS) or infusion techniques.

As used herein “liposomes” is meant to describe amphiphlic lipid bilayered structures enclosing an aqueous or hydrophilic center in which the lipid “tails” of form a bilayered envelope and the more hydrophilic “heads” are aligned at the internal and external interfaces of the media. Typical phospholipids are phospholipids or cholesterol LIPOSOMES are very simple structures consisting of one or more lipid bilayers of amphiphilic lipids, i.e. phospholipids or cholesterol. Both hydrophilic and hydrophobic drugs can be carried in liposomes, in the core or in the bilayers, respectively, or negatively charged agents can be carried by cationic liposomes, for example. The term liposome can be used to refer to liposomes in the size range of 20 nm to few μm.

As used herein “mixed micelles” are an aggregate of particles of surfactant dispersed in a liquid colloid. Exemplary detergent or surfactant structures can be aggregations of bile salts, phospholipids, tri, di- and monoglycerides, fatty acids, free cholesterol and fat soluble micronutrients. A micellar solution is a thermodynamically stable system formed spontaneously in water and organic solvents. The interaction between micelles and hydrophobic/lipophilic drugs leads to the formation of mixed micelles (MM).

As used herein, the term “lipid microparticles” is meant to include lipid nano- and microspheres. Microspheres are generally defined as small spherical particles made of any material which are sized from about 0.2 to 100 μm. Smaller spheres below 200 nm are usually called nanospheres. Lipid microspheres are homogeneous oil/water microemulsions similar to commercially available fat emulsions, and are prepared by an intensive sonication procedure or high pressure emulsifying methods (grinding methods).

The term “polymeric nanoparticles” is meant to refer to an active agent such as caspase-6 inhibitor either dissolved in a nano-polymetric matrix or entrapped or adsorbed onto a particle surface. Polymers suitable for the preparation of organic nanoparticles include cellulose derivatives and polyesters such as poly(lactic acid), poly(glycolic acid) and their copolymer. Due to their small size, their large surface area/volume ratio and the possibility of functionalization of the interface, polymeric nanoparticles are an advantageous carrier and release system. If the particle size is below about 50 nm, the particles can avoid an immune response and are better able to deliver a drug across a membrane barrier.

Various water-soluble polymers can be used in the disclosed compositions. Such polymers include, but are not limited to polyethylene oxide (PEO), ethylene oxide-propylene oxide co-polymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose, hydroxymethyl cellulose and hydroxypropyl methylcellulose, sodium carboxymethyl cellulose, methylcellulose, hydroxyethyl methylcellulose, hydroxypropyl methylcellulose, polyacrylates such as carbomer, polyacrylamides, polymethacrylamides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin or the like.

In certain embodiments in which a delayed release, or lower in the gastro-intestinal tract, at least the delayed release layer can include one or more polymers such as an acrylic polymer, acrylic copolymer, methacrylic polymer or methacrylic copolymer, including but not limited to EUDRAGIT® L100, EUDRAGIT® L100-55, EUDRAGIT® L 30 D-55, EUDRAGIT® S100, EUDRAGIT® 4135F, EUDRAGIT® RS, acrylic acid and methacrylic acid copolymers, methyl methacrylate, methyl methacrylate copolymers, ethoxyethyl methacrylates, cyanoethyl methacrylate, aminoalkyl methacrylate copolymer, polyacrylic acid, polymethacrylic acid, methacrylic acid alkylamine copolymer, polymethyl methacrylate, polymethacrylic acid anhydride, polymethacrylate, polyacrylamide, polymethacrylic acid anhydride and glycidyl methacrylate copolymers, an alkylcellulose such as ethylcellulose, methylcellulose, calcium carboxymethyl cellulose, certain substituted cellulose polymers such as hydroxypropyl methylcellulose phthalate, and hydroxypropyl methylcellulose acetate succinate, cellulose acetate butyrate, cellulose acetate phthalate, and cellulose acetate trimaleate, polyvinyl acetate phthalate, polyester, waxes, shellac, zein, or the like.

Eudragits are well known polymers and copolymers useful for controlled release applications. The EUDRAGIT® grades for enteric coatings are based on anionic polymers of methacrylic acid and methacrylates. They contain —COOH as a functional group. They dissolve at ranges from pH 5.5 to pH 7. EUDRAGIT® FS 30 D is the aqueous dispersion of an anionic copolymer based on methyl acrylate, methyl methacrylate and methacrylic acid. It is insoluble in acidic media, but dissolves by salt formation above pH 7.0. EUDRAGIT® L100-55 and L30-55 dissolve at pH above 5.5. EUDRAGIT® L100 and S100 dissolve at pH above 6.0.

Sustained-release EUDRAGIT® formulations are employed for many oral dosage forms to enable time-controlled release of active ingredients. Drug delivery can be controlled throughout the whole gastro-intestinal tract for increased therapeutic effect and patient compliance. Different polymer combinations of EUDRAGIT® RL (readily permeable) and RS (sparingly permeable) grades allow custom-tailored release profiles and enable a wide range of alternatives to achieve the desired drug delivery performance. The EUDRAGIT® NE polymer is a neutral ester dispersion which requires no plasticizer and is particularly suitable for granulation processes in the manufacture of matrix tablets and sustained release coatings.

As used herein, “osmotic agents” is intended to mean a compound that absorbs water from the environment, often used to cause swelling and expulsion of an active ingredient from a formulation. Exemplary osmagents or osmotic agents include organic and inorganic compounds such as salts, acids, bases, chelating agents, sodium chloride, lithium chloride, magnesium chloride, magnesium sulfate, lithium sulfate, potassium chloride, sodium sulfite, calcium bicarbonate, sodium sulfate, calcium sulfate, calcium lactate, d-mannitol, urea, tartaric acid, raffinose, sucrose, alpha-d-lactose monohydrate, glucose, combinations thereof and other similar or equivalent materials which are widely known in the art.

As used herein, the term “disintegrant” is intended to mean a compound used in solid dosage forms to promote the disruption of a solid mass (layer) into smaller particles that are more readily dispersed or dissolved. Exemplary disintegrants include, by way of example and without limitation, starches such as corn starch, potato starch, pre-gelatinized and modified starches thereof, sweeteners, clays, bentonite, microcrystalline cellulose (e.g., Avicel), carboxymethylcellulose calcium, croscarmellose sodium, alginic acid, sodium alginate, cellulose polyacrilin potassium (e.g., Amberlite™), alginates, sodium starch glycolate, gums, agar, guar, locust bean, karaya, pectin, tragacanth, crospovidone and other materials known to one of ordinary skill in the art. A superdisintegrant is a rapidly acting disintegrant. Exemplary superdisintegrants include crospovidone and low substituted HPC.

In certain embodiments, a plasticizer is also included in an oral dosage form. Plasticizers suitable for use in the present compositions include, but are not limited to, low molecular weight polymers, oligomers, copolymers, oils, small organic molecules, low molecular weight polyols having aliphatic hydroxyls, ester-type plasticizers, glycol ethers, polypropylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, acetyl triethyl citrate, tributyl citrate and allyl glycolate.

The compositions of the present disclosure can also include one or more functional excipients such as lubricants, thermal lubricants, antioxidants, buffering agents, alkalinizing agents, binders, diluents, sweeteners, chelating agents, colorants, flavorants, surfactants, solubilizers, wetting agents, stabilizers, hydrophilic polymers, hydrophobic polymers, waxes, lipophilic materials, absorption enhancers, preservatives, absorbents, cross-linking agents, bioadhesive polymers, retardants, pore formers, and fragrance.

Lubricants or thermal lubricants useful in the disclosed compositions include, but are not limited to fatty esters, glyceryl monooleate, glyceryl monostearate, wax, carnauba wax, beeswax, vitamin E succinate, stearate salts such as magnesium or calcium stearate, calcium hydroxide, talc, sodium stearyl fumarate, hydrogenated vegetable oil, stearic acid, glyceryl behapate, magnesium, calcium and sodium stearates, stearic acid, talc, boric acid, sodium benzoate, sodium acetate, sodium chloride, DL-leucine, polyethylene glycols, sodium oleate, or sodium lauryl sulfate.

As used herein, the term “antioxidant” is intended to mean an agent that inhibits oxidation and thus is used to prevent the deterioration of preparations by oxidation due to the presence of oxygen free radicals or free metals in the composition. Such compounds include, by way of example and without limitation, ascorbic acid, ascorbyl palmitate, butylated hydroxyanisole (BHA), butylated hydroxytoluene (BHT), hypophophorous acid, monothioglycerol, sodium ascorbate, sodium formaldehyde sulfoxylate and sodium metabisulfite and others known to those of ordinary skill in the art. Other suitable antioxidants include, for example, vitamin C, sodium bisulfite, vitamin E and its derivatives, propyl gallate or a sulfite derivative.

Binders suitable for use in the disclosed compositions include beeswax, carnauba wax, cetyl palmitate, glycerol behenate, glyceryl monostearate, glyceryl palmitostearate, glyceryl stearate, hydrogenated castor oil, microcrystalline wax, paraffin wax, stearic acid, stearic alcohol, stearate 6000 WL1644, gelucire 50/13, poloxamer 188, and polyethylene glycol (PEG) 2000, 3000, 6000, 8000, 10000 or 20000.

A buffering agent is used to resist change in pH upon dilution or addition of acid or alkali. Such compounds include, by way of example and without limitation, potassium metaphosphate, potassium phosphate, monobasic sodium acetate and sodium citrate anhydrous and dihydrate, salts of inorganic or organic acids, salts of inorganic or organic bases, and others known to those of ordinary skill in the art,

As used herein, the term “alkalizing agent” is intended to mean a compound used to provide alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art.

Exemplary binders for use in the disclosed compositions include: polyethylene oxide; polypropylene oxide; polyvinylpyrrolidone; polyvinylpyrrolidone-co-vinylacetate; acrylate and methacrylate copolymers; polyethylene; polycaprolactone; polyethylene-co-polypropylene; alkylcelluloses and cellulosic derivatives such as low substituted HPC (L-HPC), methylcellulose; hydroxyalkylcelluloses such as hydroxymethylcellulose, hydroxyethylcellulose, hydroxypropylcellulose, and hydroxybutylcellulose; hydroxyalkyl alkylcelluloses such as hydroxyethyl methylcellulose and hydroxypropyl methylcellulose; starches, pectins; PLA and PLGA, polyesters (shellac), wax such as carnauba wax, beeswax; polysaccharides such as cellulose, tragacanth, gum arabic, guar gum, and xanthan gum.

Exemplary chelating agents include EDTA and its salts, alphahydroxy acids such as citric acid, polycarboxylic acids, polyamines, derivatives thereof, and others known to those of ordinary skill in the art.

As used herein, the term “colorant” is intended to mean a compound used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide, red, other FD & C dyes and natural coloring agents such as grape skin extract, beet red powder, beta carotene, annato, carmine, turmeric, paprika, and other materials known to one of ordinary skill in the art. The amount of coloring agent used will vary as desired.

As used herein, the term “flavorant” is intended to mean a compound used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leaf oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors that have been found to be particularly useful include commercially available orange, grape, cherry and bubble gum flavors and mixtures thereof. The amount of flavoring may depend on a number of factors, including the organoleptic effect desired. Flavors will be present in any amount as desired by those of ordinary skill in the an;. Particular flavors are the grape and cherry flavors and citrus flavors such as orange.

Suitable surfactants include Polysorbate 80, sorbitan monooleate, polyoxymer, sodium lauryl sulfate or others known in the art. Soaps and synthetic detergents may be employed as surfactants. Suitable soaps include fatty acid alkali metal, ammonium, and triethanolamine salts. Suitable detergents include cationic detergents, for example, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl β-aminopropionates and 2-alkylimidazoline quaternary ammonium salts; and mixtures thereof.

A wetting agent is an agent that decreases the surface tension of a liquid. Wetting agents would include alcohols, glycerin, proteins, peptides water miscible solvents such as glycols, hydrophilic polymers Polysorbate 80, sorbitan monooleate, sodium lauryl sulfate, fatty acid alkali metal, ammonium, and triethanolamine salts, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl β-aminopropionates and 2-alkylimidazoline quaternary ammonium salts; and mixtures thereof.

Solubilizers include cyclodextrins, povidone, combinations thereof, and others known to those of ordinary skill in the art.

Exemplary waxes include carnauba wax, beeswax, microcrystalline wax and others known to one of ordinary skill in the art.

Exemplary absorption enhancers include dimethyl sulfoxide, Vitamin E PGS, sodium cholate and others known to one of ordinary skill in the art.

Preservatives include compounds used to prevent the growth of microorganisms. Suitable preservatives include, by way of example and without limitation, benzalkonium chloride, benzethonium chloride, benzyl alcohol, cetylpyridinium chloride, chlorobutanol, phenol, phenylethyl alcohol, phenylmercuric nitrate and thimerosal and others known to those of ordinary skill in the art.

Examples of absorbents include sodium starch glycolate (Explotab™, Primojel™) and croscarmellose sodium (Ac-Di-Sol™), cross-linked PVP (Polyplasdone™ XL 10), veegum, clays, alginates, PVP, alginic acid, carboxymethylcellulose calcium, microcrystalline cellulose (e.g., Avicel), polacrillin potassium (e.g., Amberlite™), sodium alginate, corn starch, potato starch, pregelatinized starch, modified starch, cellulosic agents, montmorrilonite clays (e.g., bentonite), gums, agar, locust bean gum, gum karaya, pectin, tragacanth, and other disintegrants known in to those of ordinary skill in the art.

A cross-linking agent is defined as any compound that will form cross-links between the moieties of the polymer. A cross-linking agent can include, by way of example and without limitation, an organic acid, an alpha-hydroxy acid, and a beta-hydroxy acid. Suitable cross-linking agents include tartaric acid, citric acid, fumaric acid, succinic acid and others known to those of ordinary skill in the art.

Bioadhesive polymers include polyethylene oxide, Klucel® (hydroxypropyl cellulose), CARBOPOL, polycarbophil, GANTREZ, Poloxamer, and combinations thereof, and others known to one of ordinary skill in the art.

Retardants are agents that are insoluble or slightly soluble polymers with a glass transition temperature (Tg) above 45° C., or above 50° C. before being plasticized by other agents in the formulation including other polymers and other excipients needed for processing. The excipients include waxes, acrylics, cellulosics, lipids, proteins, glycols, and the like.

Exemplary pore formers include water-soluble polymers such as polyethylene glycol, propylene glycol, poloxamer and povidone; binders such as lactose, calcium sulfate, calcium phosphate and the like; salts such as sodium chloride, magnesium chloride and the like; combinations thereof and other similar or equivalent materials which are widely known in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

It should be understood that compounds used in the art of pharmaceutical formulation generally serve a variety of functions or purposes. Thus, if a compound named herein is mentioned only once or is used to define more than one term herein, its purpose or function should not be construed as being limited solely to that or those named purpose(s) or function(s).

### BRIEF DESCRIPTION OF THE DRAWINGS

The following drawings form part of the present specification and are included to further demonstrate certain aspects of the present inventions. The disclosure can be better understood by reference to one or more of these drawings in combination with the detailed description of specific embodiments presented herein.

FIG. 1A shows the synthesis scheme for compounds 8a-f FIG. 1B shows possible tautomeric forms of compounds 8a-f.

FIG. 2 shows the hydrolytic ring opening of compound 8 to compound 9 and decarboxylation of compound 9e to compound 10.

FIG. 3 and FIGS. 3a-h show the structure of the PG3 compounds and its analogs.

FIG. 4 shows that the PG3 compounds inhibit the caspase 6 enzyme in a dose dependent fashion. Different concentrations of the PG3 compounds were incubated with the Htt protein and the caspase-6 enzyme. The Htt substrate from COS-7 cell lysates is cleaved by caspase 6 and fragments are detected by FRET between the N-terminal BKP1 antibody and the neo-epitope antibody against amino acid 586. (A) shows the dose response curves for compound PG3, PG3a and PG3b (B) shows the dose response curves for compound PG3c, PG3d and PG3e (C) shows the dose response curves for compound PG3f, PG3g and PG3h.

FIG. 5 shows that the presence of the PG3d compound inhibits the cleavage of Htt by caspase-6 in a Western blot assay. COS-7 cells co-transfected with the htt-4C construct and the human caspase-6 lacking the pro-domain that were either left untreated, treated with 10 uM of the PG3d compound or treated with 3 uM of the pan-caspase inhibitor (Q-VD-Oph). (A-upper panel) The cleavage of the Htt protein to generate the 586 amino acid fragment was analyzed by Western blot using the pan-HTT BKP1 antibody. The presence of the PG3d inhibitor or the pan caspase inhibitor resulted in a reduced cleavage of the Htt protein. (A lower panel) The presence of the PG3d compound or the pan-caspase inhibitor (Q-VD-Oph) also resulted in a reduced level of active caspase-6 enzyme. (B) shows a graphical representation of the level of the 586 amino acid fragment compared to actin levels from the western blot analysis. Protein levels were quantified using Licor Odyssey imaging software. The expression of the Htt-586 fragment relative to actin expression in shown on the y-axis. The presence of 10 uM of the PG3d compound resulted in a significant reduction in the expression of the Htt cleavage fragment as compared to untreated cells. Student's t test **: p<0.01

FIG. 6 shows that the PG3 compounds inhibit the cleavage of lamin A in HEK293 cells by caspase-6 as quantified by the Mesoscale ELISA method. (A) shows the dose response curves for compounds PG3a, PG3b, PG3d and PG3d. (B) shows the dose response curves for compounds PG3e, PG3g and PG3h.

FIG. 7 shows that PG3d inhibit the intraneuronal activation of Caspase-6. Primary cortical neurons from FVB/N mice were treated with 10 uM camptothecin for 30 h in the presence or absence of 10 uM PG3d. Camptothecin treatment leads to the activation of caspase-6, which can be quantified by measuring the cleavage of lamin A (Ehrnhoefer et al. PLoS One, 2011. 6(11):e27680). The presence of PG3d in the neuronal medium significantly reduces caspase-6 activity. Student's t-test, **: p<0.01.

FIG. 8 shows that PG3d improves the neuronal viability during excitotoxic stress. Primary cortical neurons from FVB/N mice were treated with different amounts of NMDA in the medium for 20 h, which leads to a loss of intracellular ATP levels, a measure of viability. The presence of 10 uM PG3d significantly improves neuronal viability in this paradigm. Two-way ANOVA: NMDA treatment p<0.0001, PG3d treatment p=0.0001. Post-hoc Bonferroni test: **=p<0.01.

FIG. 9 is the results of cells studies demonstrating the interaction of caspase-6 with Htt fragment in COS-7 cells. Following transfection, the COS-7 cells were exposed to either DMSO, the pan-caspase inhibitor Q-VD-Oph (Q-VD-OPh) or the PG3d compound (PG3d). After a 24 hr incubation period, cell lysates were either processed by Western blot or were immunoprecipitated with Caspase-6 antibody followed by Western blot analysis. (A) Western blot analysis of the lysates from co-transfected COS-7 cells that were either treated with DMSO (n.t), exposed to 3 uM of the pan-caspase inhibitor Q-VD-Oph (Q-VD-OPh) or were treated with 10 uM of the PG3d compound (PG3d). The upper panel shows Western blot analysis with the Htt antibody 2166 (Millipore). The lower panel shows Western blot analysis with Caspase-6 antibody HD91 to detect the full length and active forms of caspase 6. (B) Western blot analysis of the cell lysates from FIG. 9A following immunoprecipitation with a caspase-6 antibody to identify the binding partners of the caspase-6 enzyme. The upper panel shows Western blot analysis with the Htt antibody 2166 (Millipore) and the lower panel shows Western blot analysis with the HD91 caspase-6 antibody.

FIG. 10 is a bar graph quantitizing co-precipitation data shown in FIG. 9B by densitometric analysis. 1way ANOVA p=0.031, post-hoc Tukey's test *: p<0.05.

### DETAILED DESCRIPTION OF THE INVENTION

### Sequences:

SEQ ID NO: 1 (htt-4C). SEQ ID NO: 1 has an additional 10 amino acids at the N-terminus (relative to wild-type huntingtin), comprising the His-tag to enable processing of the expressed polypeptide. Htt-4C is truncated at amino acid 1212 (numbering according to the wild-type huntingtin sequence, and has four D to A amino acid substitutions at amino acids 513, 530, 552 and 589 (numbering according to the wild-type huntingtin sequence) marked by bold, underlined text. The IVLD Caspase-6 cleavage site is marked with a double-underline.

(SEQ ID NO: 1)
MHHHHHHEFPMATLEKLMKAFESLKSFQQQQQQQQQQQQQQQQQPP
PPPPPPPPPQLPQPPPQAQPLLPQPQPPPPPPPPPPGPAVAEEPLH
RPKKELSATKKDRVNHCLTICENIVAQSVRNSPEFQKLLGIAMELF
LLCSDDAESDVRMVADECLNKVIKALMDSNLPRLQLELYKEIKKNG
APRSLRAALWRFAELAHLVRPQKCRPYLVNLLPCLTRTSKRPEESV
QETLAAAVPKIMASFGNFANDNEIKVLLKAFIANLKSSSPTIRRTA
AGSAVSICQHSRRTQYFYSWLLNVLLGLLVPVEDEHSTLLILGVLL
TLRYLVPLLQQQVKDTSLKGSFGVTRKEMEVSPSAEQLVQVYELTL
HHTQHQDHNVVTGALELLQQLFRTPPPELLQTLTAVGGIGQLTAAK
EESGGRSRSGSIVELIAGGGSSCSPVLSRKQKGKVLLGEEEALEDD
SESRSDVSSSALTASVKDEISGELAASSGVSTPGSAGHDIITEQPR
SQHTLQADSVALASCDLTSSATDGDEEAILSHSSSQVSAVPSDPAM
DLNAGTQASSPISDSSQTTTEGPDSAVTPSDSSEIVLDGTANQYLG
LQIGQPQDEDEEATGILPDEASEAFRNSSMALQQAHLLKNMSHCRQ
PSDSSVDKFVLRDEATEPGDQENKPCRIKGDIGQSTDDDSAPLVHC
VRLLSASFLLTGGKNVLVPDRDVRVSVKALALSCVGAAVALHPESF
FSKLYKVPLDTTEYPEEQYVSDILNYIDHGDPQVRGATAILCGTLI
CSILSRSRFHVGDWMGTIRTLTGNTFSLADCIPLLRKTLKDESSVT
CKLACTAVRNCVMSLCSSSYSELGLQLIIDVLTLRNSSYWLVRTEL
LETLAEIDFRLVSFLEAKAENLHRGAHHYTGLLKLQERVLNNVVIH
LLGDEDPRVRHVAAASLIRLVPKLFYKCDQGQADPVVAVARDQSSV
YLKLLMHETQPPSHFSVSTITRIYRGYNLLPSITDVTMENNLSRVI
AAVSHELITSTTRALTFGCCEALCLLSTAFPVCIWSLGWHCGVPPL
SASDESRKSCTVGMATMILTLLSSAWFPLDLSAHQDALILAGNLLA
ASAPKSLRSSWASEEEANPAATKQEEVWPALGDRALVPMVEQLFSH
LLKVINICAHVLDDVAPGPAIKAALPSLTNPPSLSPIRRKGKEKEP
GEQASVPLSPKKGSEASAASRVEGYPYDVPDYA

SEQ ID NO: 2 (caspase-6 delta prodomain). SEQ ID NO: 2 comprises amino acids 24-293 of human caspase-6, with the prodomain (aa 1-23) deleted. This deletion leads to faster intracellular auto-activation of the enzyme after transfection (Klaiman et al, BBA, 2009. 1793(3): 592-601). The protein has additional 31 amino acids at the C-terminus (relative to wild-type caspase-6), comprising the DDK-tag to enable detection of the expressed polypeptide.

(SEQ ID NO: 2)
MAFYKREMFDPAEKYKMDHRRRGIALIFNHERFFWHLTLPERRGTC
ADRDNLTRRFSDLGFEVKCFNDLKAEELLLKIHEVSTVSHADADCF
VCVFLSHGEGNHIYAYDAKIEIQTLTGLFKGDKCHSLVGKPKIFII
QACRGNQHDVPVIPLDVVDNQTEKLDTNITEVDAASVYTLPAGADF
LMCYSVAEGYYSHRETVNGSWYIQDLCEMLGKYGSSLEFTELLTLV
NRKVSQRRVDFCKDPSAIGKKQVPCFASMLTKKLHFFPKSNTRTPL
EQKLISEEDLAAMISWITRDDDDKV

SEQ ID NO: 3 (wt htt). SEQ ID NO: 3 has an additional 10 amino acids at the N-terminus (relative to wild-type huntingtin), comprising the His-tag to enable processing of the expressed polypeptide. Wt Htt is truncated at amino acid 1212 (numbering according to the wild-type huntingtin sequence.

(SEQ ID NO: 3)
MHHHHHHEFPMATLEKLMKAFESLKSFQQQQQQQQQQQQQQQQQPP
PPPPPPPPPQLPQPPPQAQPLLPQPQPPPPPPPPPPGPAVAEEPLH
RPKKELSATKKDRVNHCLTICENIVAQSVRNSPEFQKLLGIAMELF
LLCSDDAESDVRMVADECLNKVIKALMDSNLPRLQLELYKEIKKNG
APRSLRAALWREAELAHLVRPQKCRPYLVNLLPCLTRTSKRPEESV
QETLAAAVPKIMASFGNFANDNEIKVLLKAFIANLKSSSPTIRRTA
AGSAVSICQHSRRTQYFYSWLLNVLLGLLVPVEDEHSTLLILGVLL
TLRYLVPLLQQQVKDTSLKGSFGVTRKEMEVSPSAEQLVQVYELTL
HHTQHQDHNVVTGALELLQQLFRTPPPELLQTLTAVGGIGQLTAAK
EESGGRSRSGSIVELIAGGGSSCSPVLSRKQKGKVLLGEEEALEDD
SESRSDVSSSALTASVKDEISGELAASSGVSTPGSAGHDIITEQPR
SQHTLQADSVALASCDLTSSATDGDEEAILSHSSSQVSAVPSDPAM
DLNAGTQASSPISDSSQTTTEGPDSAVTPSDSSEIVLDGTANQYLG
LQIGQPQDEDEEATGILPDEASEAFRNSSMALQQAHLLKNMSHCRQ
PSDSSVDKFVLRDEATEPGDQENKPCRIKGDIGQSTDDDSAPLVHC
VRLLSASELLTGGKNVLVPDRDVRVSVKALALSCVGAAVALHPESF
FSKLYKVPLDTTEYPEEQYVSDILNYIDHGDPQVRGATAILCGTLI
CSILSRSRFHVGDWMGTIRTLTGNTFSLADCIPLLRKTLKDESSVT
CKLACTAVRNCVMSLCSSSYSELGLQLIIDVLTLRNSSYWLVRTEL
LETLAEIDFRLVSFLEAKAENLHRGAHHYTGLLKLQERVLNNVVIH
LLGDEDPRVRHVAAASLIRLVPKLFYKCDQGQADPVVAVARDQSSV
YLKLLMHETQPPSHFSVSTITRIYRGYNLLPSITDVTMENNLSRVI
AAVSHELITSTTRALTFGCCEALCLLSTAFPVCIWSLGWHCGVPPL
SASDESRKSCTVGMATMILTLLSSAWFPLDLSAHQDALILAGNLLA
ASAPKSLRSSWASEEEANPAATKQEEVWPALGDRALVPMVEQLFSH
LLKVINICAHVLDDVAPGPAIKAALPSLTNPPSLSPIRRKGKEKEP
GEQASVPLSPKKGSEASAASRVEGYPYDVPDYA

SEQ ID NO: 4 (full-length caspase-6). SEQ ID NO: 4 comprises amino acids 1-293 of human caspase-6. The protein has additional 31 amino acids at the C-terminus (relative to wild-type caspase-6), comprising the DDK-tag to enable detection of the expressed polypeptide.

(SEQ ID NO: 4)
MSSASGLRRGHPAGGEENMTETDAFYKREMFDPAEKYKMDHRRRGIA
LIFNHERFFWHLTLPERRGTCADRDNLTRRFSDLGFEVKCFNDLKAE
ELLLKIHEVSTVSHADADCFVCVFLSHGEGNHIYAYDAKIEIQTLTG
LFKGDKCHSLVGKPKIFIIQACRGNQHDVPVIPLDVVDNQTEKLDTN
ITEVDAASVYTLPAGADFLMCYSVAEGYYSHRETVNGSWYIQDLCEM
LGKYGSSLEFTELLTLVNRKVSQRRVDFCKDPSAIGKKQVPCFASML
TKICLHFFPKSNTRTPLEQKLISEEDLAAMISWITRDDDDKV

### Cell Transfection and Cell Lysis

COS-7 cells were grown in DMEM supplemented with 10% fetal bovine serum, 1%/penicillin/streptomycin and 0.5% glutamine. Transfections were performed using the Fugene reagent (Roche) according to manufacturer's instructions. The transfected DNA encodes amino acids 1-1212 of the human Htt protein with 15 glutamines and a C-terminal HA-tag, under control of a CMV promoter (Warby, 2008, supra; Wellington et al., 2000. J Biol Chem 275:19831-19838). The construct that was transfected is 4c Htt, which contains D→A mutations at amino acids 513, 530, 552 and 589 (SEQ ID NO: 1). 24 h after transfection, cells were harvested by trypsinization, pellets washed in PBS and lysed by suspension in lysis buffer (50 mM Tris pH 8, 150 mM NaCl, 1% Igepal, supplemented with 1× complete protease inhibitor (Roche) and 4 mM Pefabloc). Lysates were incubated on ice for 10 min, vortexed and sonicated for 4 sec before centrifugation at 21,000×g for 10 min at 4° C. Supernatants were saved, protein concentration determined with the Biorad DC assay and stored at −80° C. until use.

HEK 293 cells were grown in DMEM supplemented with 10% fetal bovine serum, 1%/penicillin/streptomycin and 0.5% glutamine. Transfections were performed using the Fugene reagent (Roche) according to manufacturer's instructions. The transfected DNA encodes amino acids 24-293 of the human caspase 6 enzyme with a C-terminal DDK tag under the control of a CMV promoter (vector pCMVSport6) (SEQ ID NO: 2). The transfected HEK 293 were used to measure the intracellular activity of caspase-6 as evidenced by cleavage of the lamin substrate (described below).

Purification of Htt from COS-7 Cell Lysates

50 μl HA-agarose beads (EZ-View, Sigma) were mixed with 1 ml lysis buffer, centrifuged at 8200×g for 30 sec and the supernatant was discarded. The beads were mixed with 200 μl cell lysate diluted to 0.5 μg/μl in lysis buffer and incubated for 2 h at 4° C. on a rotating wheel. An aliquot of the diluted cell lysate was saved as the input fraction. The sample was centrifuged at 8200×g for 30 sec and the supernatant was saved, the beads were washed three times with 100 μl lysis buffer and supernatants were saved as wash fractions. Elution was performed by adding 100 μl HA peptide (100 μg/ml, Sigma) in RIPA buffer to the beads (50 mM Tris pH 8, 150 mM NaCl, 1% Igepal, 0.5% Na-deoxycholate, 0.1% SDS) and incubating for 10 min at 37° C. The elution step was repeated 5 times, all eluates were saved. The beads were then mixed with SDS loading dye and after heat denaturation run together with 10 μl aliquots of all fractions on a 3-8% NuPage Tris-Acetate gel (Invitrogen). Gels were either blotted for detection with the BKP1 (Kalchman et al, J Biol Chem (1996), 271 (32):19385)) and HA antibodies with the Odyssey imaging system (Li-cor Biosciences) or stained with Coomassie dye for detection of total protein.

### Assessment of Htt Cleavage by Western Blot

Aliquots of COS-7 lysates expressing 4c Htt corresponding to 25 μg protein were diluted with cleavage buffer (50 mM HEPES pH 7.4, 100 mM NaCl, 0.1% CHAPS, 1 mM EDTA, 10% glycerol) and the desired amount of recombinant caspase-6 (BioMol) to 10 μl. For experiments including caspase-6 inhibitor compounds, the desired amount of inhibitor was mixed with the caspase before addition to the COS-7 lysate. Samples were incubated for 1 h at 37° C. and analyzed on a 3-8% NuPage Tris-Acetate gel (Invitrogen), followed by Western blotting and detection with the BKP1 and neo-586 antibodies with the Odyssey imaging system (Li-cor Biosciences). The BKP1 and neo-586 antibodies have been described previously (Warby et al. Hum. Mol. Genet 2008. 17(15):2390-404.).

### Quantification of Htt Expression by FRET

Dilution series of cell lysates were prepared in sample buffer (1× PBS without CaCl2 or MgCl2, 0.4% Triton, 1× complete protease inhibitor cocktail (Roche)), Tb-labelled BKP1 antibody and D2-labelled HA antibody (Cisbio) were diluted to 1 ng/μl (Tb) and 10 ng/μl (D2) in antibody dilution buffer (50 mM NaH2PO4, 0.1% BSA, 0.05% Tween). Antibodies were pre-mixed at a 1:1 ratio, then 10 μl cell lysate and 2 μl antibody mix were pipetted into each well of a white 384 well plate (Nunc). The plate was centrifuged briefly and FRET was measured on a Victor 3 multilabel plate reader (Perkin Elmer) with the following settings: Excitation: 340 nm, Emission 1: 615 nm, Emission 2: 665 nm, 50 μs delay, 200 μs window time, 2000 μs cycle time. To obtain the final FRET signal, the ratio between Emission 2/Emission 1 (D2/Tb signal) was calculated.

### Simultaneous Caspase-6 Cleavage and 586 Fragment Detection by FRET

Dilution series of cell lysates and caspase-6 (2.2× final concentration) were prepared in FRET cleavage buffer (10 mM HEPES pH 7.4, 100 mM NaCl, 0.05% gelatin, 0.1% CHAPS, 2 mM DTT) since this buffer was previously found to best stabilize caspase-6 in dilute form at room temperature. For negative controls, 22 μM zVAD-fmk was added to the caspase-6 dilutions.

Tb-labelled BKP1 antibody and D2-labelled 586 antibody (Cisbio) were diluted to 1 ng/μl (Tb) and 10 ng/μl (D2) in FRET cleavage buffer and pre-mixed at a 1:1 ratio.

In each well of a white 384 well plate (Nunc), 10 μl cell lysate were mixed with 10 μl caspase and 2 μl antibody mix, the plate was centrifuged briefly and incubated in the Victor 3 multilabel plate reader (Perkin Elmer) at 37° C. FRET was measured every 30 min for up to 2 h with the following settings: Excitation: 340 nm, Emission 1: 615 nm, Emission 2: 665 nm, 50 μs delay, 200 μs window time, 2000 μs cycle time. To obtain the final FRET signal, the ratio between Emission 2/Emission 1 (D2/Tb signal) was calculated. After 2 h at 37° C., the assay plate was sealed and after further incubation at 4° C. for 20 h, the FRET signal was read again.

### Assessment of Intracellular Htt Cleavage by Western Blot

COS-7 cells were co-transfected with the 4c htt fragment and the human caspase-6 lacking the pro-domain which leads to fast autoactivation of the caspase-6 enzyme and the generation of the 586 aa Htt cleavage fragment. The co-transfected cells were exposed to either 10 uM of the PG3d compound, 3 uM of the Q-VD-Oph pan-caspase inhibitor or were left untreated. Non-transfected cells were included as a negative control and the purified 586 aa Htt fragment was included as a positive control. Cell lysates were subjected to Western blotting and the 586AA fragment generated intracellularly was detected with Htt antibody 2166 (Millipore). Caspase-6 expression and activation was assessed by Western blotting using antibody HD91 (Ehrnhoefer et al, HMG, 2014. 23(3):717-29).

### Assessment of Lamin Cleavage in HEK 293 Cells Overexpressing Caspase-6

The quantitative assessment of intracellular lamin cleavage in caspase-6 transfected HEK 293 cells was done as described previously (Ehrnhoefer et al. PLoS One, 2011. 6(11):e27680) Briefly, HEK 293 cell lysates were adjusted to 1 μg protein/μl in lysis buffer, diluted to 0.2 μg/μl in PBS and 5 μl were added to a Multi-Array high-bind 96 well plate (Mesoscale discovery). After incubation at room temperature for 1 h, the wells were blocked by adding 150 μl 5% BSA in PBS, followed by further incubation at room temperature for 1 h. Wells were then washed 3× with 150 μl PBS+0.05% Tween, and 25 μl antibody mix was added (Cell signaling #2036 at 1:100 dilution, Mesoscale discovery goat-anti-rabbit sulfo-tag at 1:500 dilution in PBS with 1% BSA). After lh incubation at room temperature, the wells were washed 3× with 150 μl PBS+0.05% Tween, and 150 μl/well 2× reading reagent (Mesoscale discovery) was added. The plate was read on a Sector imager 6000 (Mesoscale discovery).

### Assessment of Lamin Cleavage in Primary Neuronal Culture

Cortical neuronal cultures from FVB mice were prepared as described (Metzler et al, J Neurosci (2007) 27(9):2298). At day 10 in vitro, cells were treated with camptothecin, and after 30 h harvested by scraping in PBS supplemented with 1× complete protease inhibitor (Roche) and 4 mM Pefabloc. Cells were lysed by suspension in lysis buffer (50 mM Tris pH8, 150 mM NaCl, 1% Igepal, supplemented with 1× complete protease inhibitor (Roche) and 4 mM Pefabloc). Lysates were incubated on ice for 10 min, vortexed and sonicated for 4 sec before centrifugation at 21 000×g for 10 min at 4° C. Supernatants were saved, protein concentration determined with the Biorad DC assay and quantitative assessment of cleaved lamin A in neuronal lysates was performed with the Mesoscale ELISA method as described in Ehrnhoefer et al. PLoS One, 2011. 6(11):e27680.

### Assessment of Neuronal Viability During Excitotoxic Stress

Cortical neuronal cultures from FVB mice were prepared as described (Metzler et al, J Neurosci (2007) 27(9):2298). At day 10 in vitro, cells treated with either 10 uM PG3d or DMSO as a negative control and then were treated with either 25 nM NMDA, 50 uM of NMDA or were left untreated for 20 hr. The measurement of intracellular ATP was used as an assessment of neuronal viability as described previously (Uribe et al, HMG 2012. 21(9):1954-67) using the Cell-titer glo kit from Promega according to manufacturer's instructions.

### Assessment of the Binding Interaction Between htt and Caspase 6.

COS-7 cells were co-transfected with the 1212 amino acid HTT fragment (SEQ ID NO: 3) and the full length human caspase-6 enzyme (SEQ ID NO:4). The co-transfected cells were exposed to either 10 uM of the PG3d compound, 3 uM of the Q-VD-Oph pan-caspase inhibitor or were treated with DMSO as a control. An aliquot of the cell lysates were subjected to Western blotting to detect HTT fragments with the Htt antibody 2166 (Millipore) and the presence of caspase-6 was detected using the HD91 antibody (Ehrnhoefer et al, HMG, 2014. 23(3):717-29). The remaining cell lysates (500 ug protein) were immunoprecipitated with 5 ug of Caspase-6 antibody for 16 hrs at 4 C. The immunoprecipitated proteins were then applied to acrylamide gels and immunoblotted to detect either the HTT fragments or the presence of the active caspase 6 enzyme using the antibodies described above.

### Synthetic Methods for the PG3 Compound and its Analogs.

As described in Table 1, 3-Phenylprop-2-ynamide (1a) was prepared in high yield according to literature procedures by the reaction of 3-phenylprop-2-ynoic acid ester with aqueous ammonia solution (Struebing et al. Tetrahedron (2005) 61:11333). Following this procedure, the corresponding arylpropynamides 1b-e were obtained in good yields by ammonolysis of the crude arylpropynoic ethyl esters, which in turn resulted from esterification of the corresponding arylpropynoic acids with ethanol.

TABLE 1
Synthesis of Arylpropynamides (1)

| R | Amide 1 | Yield (%) |
|---|---|---|
| Ph | 1a | 92 |
| 2-ClC₆H₄ | 1b | 72 |
| 4-ClC₆H₄ | 1c | 78 |
| 4-BrC₆H₄ | 1d | 76 |
| 4-O₂NC₆H₄ | 1e | 67 |

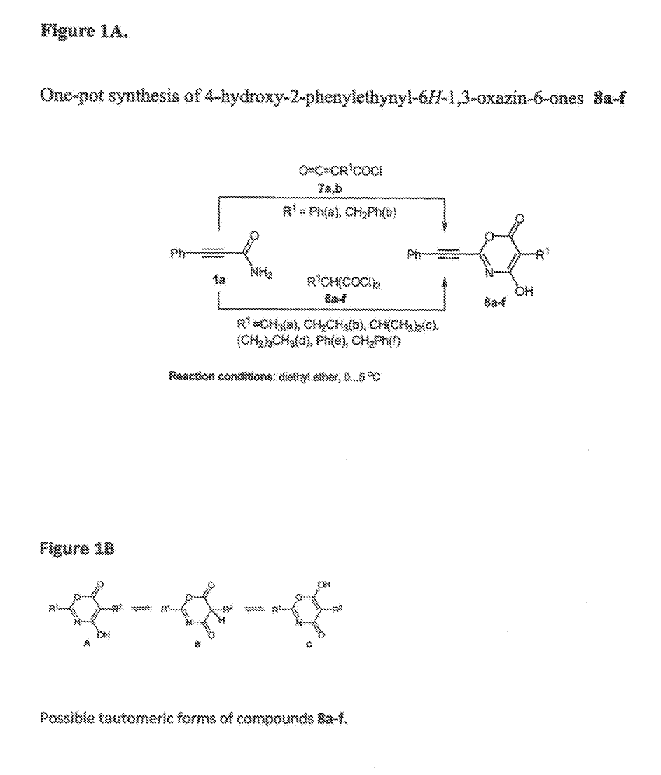

Treatment of 3-phenylprop-2-ynamide (1a) with monosubstituted malonyl chlorides (6a-f) or (chlorocarbonyl)ethylketenes (7a,b) in diethyl ether at 0-5° C. delivered the 4-hydroxy-2-(phenylethynyl)-6H-1,3-oxazin-6-ones (8a-f) in 61-87% yield (as shown in Table 2 and FIG. 1A).

Theoretically, the heterocycles 8 (R1=C≡CPh) can adopt the tautomeric forms A-C (FIG. 1B). Mass spectra of similar 4-hydroxy-6H-1,3-oxazin-6-ones (R1=aryl; R2=H, Me) showed the presence of 4-hydroxy-6-oxa, dipolar-ionic and dioxo forms mixture in the gas phase. The presence of a substituent in the para position of the benzene ring has a strong influence on the tautomeric ratio. The quantum-chemical calculations revealed structure A as the most favorable 1,3-oxazine tautomer in the gas phase. Dissolved in tetrahydrofuran and dimethyl sulfoxide-d6 these compounds predominantly exist as tautomerA. (Stanovnik et al. Adv. Heterocycl Chem (2006) 61:1; Zakhs et al. Khim. Geterotsikl. Soedin (1990) 552; Chem. Abstr (1990) 113: 211247; Zakha et al. Khim Geterotsikl (1987) 386: Chem. Abstr. (1988) 108:5938) 1 In the case of 4-hydroxy-6H-1,3-oxazin-6-ones (R1=Bn, Me and R2=Ph) Sheibani et al were able to detect the tautomer B along with the major tautomer A by NMR spectroscopy. (Sheibani et al. ARKIVOC (2005) (xv), 88).

The ¹H and ¹³C NMR spectra of the herein described 4-hydroxy-2-(phenylethynyl)-6H-1,3-oxazin-6-ones (8a-f), exhibited only signals consistent with the tautomer A, which in the case of 8d could unambiguously be proven by X-ray crystal structure analysis.

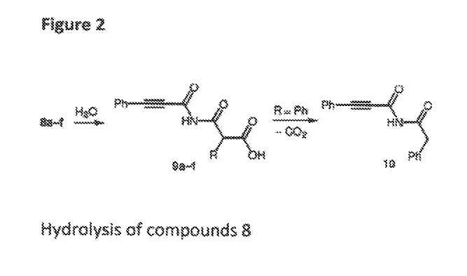

Prepared 4-hydroxy-2-(phenylethynyl)-6H-1,3-oxazin-6-ones 8a-f have been proved to be unstable in dimethyl sulfoxide solution. The NMR spectra of samples of 8a-f that had been kept in dimethyl sulfoxide-d6 solution for 24 hours at ambient temperature showed in addition to the signals for 1,3-oxazin-6-ones 8a-f, an additional set of signals belonging to hydrolysis products 9a-f (FIG. 2). In the case of 8e the intermediate 9e underwent spontaneous decarboxylation giving imide 10 as the final product (FIG. 2); imide 10 was also obtained on treatment of 4-hydroxy-5-phenyl-2-(phenylethynyl)-6H-1,3-oxazin-6-one (8e) with boiling water. The signals of isolated imide 10 are identical to additional signals in the NMR spectra of 8e that has been kept in dimethyl sulfoxide-d6 solution According to these results, it can be concluded that 1,3-oxazin-6-ones 8a-f are hydrolyzed by traces of water in dimethyl sulfoxide solution (FIG. 2).

TABLE 2
One-Pot Synthesis of 4-Hydroxy-5-phenyl-2-(phenylethynyl)-6H-
1,3-oxazin-6-ones 8 ^{a}

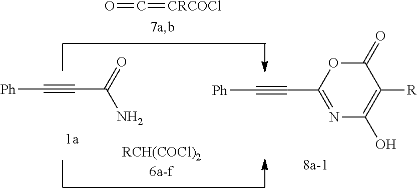

| Product | R | Yield (%) |
|---|---|---|
| 8a | Me | 74 |
| 8b | Et | 70 |
| 8c | i-Pr | 62 |
| 8d | Bu | 65 |
| 8e | Ph | 87 |
| 8f | Bn | 61 |

^{a} Reaction conditions: Et₂O, 0-5° C.

### 3-Phenylprop-2-ynamide (1a)

Prepared from ethyl phenylpropynoate (8.71 g, 8.26 mL, 50 mmol) according to the literature procedure as colorless crystals; (Struebing et al. Tetrahedron (2005) 61:11333) ] yield: 6.70 g (92%); mp 100-102° C.

IR (KBr): 3383 and 3179 (NH₂), 2223 (C≡C), 1654 cm⁻¹ (C═O).

¹H NMR (400 MHz, DMSO-d₆): δ=8.18 (s, 1H, NH₂), 7.71 (s, 1H, NH₂), 7.57 (m, 2H, o-CH_{Ar}), 7.51 (m, 1H, p-CH_{Ar}), 7.46 (m, 2H, m-CH_{Ar}).

¹³C NMR (100 MHz, DMSO-d₆): δ=153.9 (C═O), 132.0 (o-CH_{Ar}), 130.2 (p-CH_{Ar}), 129.0 (m-CH_{Ar}), 119.9 (i-C_{Ar}), 84.2 (C≡C), 82.9 (C≡C).

MS (EI, 70 eV): m/z (%)=145 (64) [M]⁺, 129 (100) [M-NH₂]⁺, 75 (13), no other peaks >10%.

Anal. Calcd for C₉H₇NO: C, 74.47; H, 4.86; N, 9.65. Found: C, 74.59; H, 4.84; N, 9.57.

### 3-Arylprop-2-ynamides (1b-e); General Procedure

The corresponding arylpropynoic acid (15 mmol) was heated under reflux with EtOH (60 mmol) in the presence of H₂SO₄ for 5 h. Excess EtOH was removed under reduced pressure and the residue was washed with H₂O and sat. aq NaHCO₃. The organic layer was separated and the aqueous layer was extracted with CHCl₃ (3×50 mL). The combined organic layers were dried (MgSO₄) and concentrated under reduced pressure to give ethyl 3-arylprop-2-ynoates as oily residues, which were subsequently dissolved in 25% aq NH₃ (60 mmol) and stirred at r.t. for 24 h. The resulting solid products 1b-d were filtered off and recrystallized.

### 3-(2-Chlorophenyl)prop-2-ynamide (1b) (Mariella et al. Can J. Chem (1965) 43:2426; Unangst et al. J. Heterocycl Chem. (1973) 10: 399)

Colorless crystals; yield: 1.94 g (72%); mp 121-122° C. (EtOH—H₂O, 1:1).

IR (KBr): 3318 and 3162 (NH2), 2225 (C≡C), 1655 cm-1 (C═O).

¹H NMR (400 MHz, DMSO-d₆): δ=8.25 (s, 1H, NH₂), 7.81 (s, 1H, NH₂), 7.69 (m, 1H, H6′), 7.62 (m, 1H, H3′), 7.52 (m, 1H, H4′), 7.43 (m, 1H, H5′).

13C NMR (100 MHz, DMSO-d6): δ=153.4 (C═O), 135.1 (C2′), 134.0 (C6′), 131.6 (C4′), 129.5 (C3′), 127.5 (C5′), 119.8 (C1′), 84.5 (C═C), 79.2 (C≡C).

MS (EI, 70 eV): m/z (%)=181/179 (13/46) [M]⁺, 165/163 (34/100) [M-NH₂]⁺, 136 (10), 99 (16), 75 (13), 74 (15), no other peaks >10%.

Anal. Calcd for C₉H₆ClNO: C, 60.19; H, 3.37; Cl, 19.74; N, 7.80. Found: C, 60.07; H, 3.38; Cl, 19.75; N, 7.87.

### 3-(4-Chlorophenyl)prop-2-ynamide (1c) (Schmitt J. FR 1305340 (1962); Chem Abstr. (1963) 58:46538)

Colorless crystals; yield: 2.10 g (78%); mp 186-188° C. (MeCN).

IR (KBr): 3399 and 3172 (NH₂), 2218 (C≡C), 1656 cm⁻¹ (C═O).

¹H NMR (400 MHz, DMSO-d₆): δ=8.21 (s, 1H, NH₂), 7.75 (s, 1H, NH₂), 7.60 (m, 2H, o-CH_{Ar}), 7.54 (m, 2H, m-CH_{Ar}).

¹³C NMR (100 MHz, DMSO-d₆): δ=153.7 (C═O), 135.0 (p-Cl—C_{Ar}), 133.8 (o-CH_{Ar}), 129.1 (m-CH_{Ar}), 118.8 (i-C_{Ar}), 85.0 (C≡C), 81.6 (C≡C).

MS (EI, 70 eV): m/z (%)=181/179 (17/53) [M]⁺, 165/163 (32/100) [M-NH₂]⁺, 99 (15), 75 (13), no other peaks >10%.

Anal. Calcd for C₉H₆ClNO: C, 60.19; H, 3.37; Cl, 19.74; N, 7.80. Found: C, 60.29; H, 3.37; Cl, 19.64; N, 7.92.

### 3-(4-Bromophenyl)prop-2-ynamide (1d)

Colorless crystals; yield: 2.55 g (76%); mp 202-204° C. (dec) (MeCN).

IR (KBr): 3386 and 3172 (NH₂), 2216 (C≡C), 1655 cm⁻¹ (C═O).

¹H NMR (400 MHz, DMSO-d₆): δ=8.23 (s, 1H, NH₂), 7.75 (s, 1H, NH₂), 7.68 (m, 2H, o-CH_{Ar}), 7.52 (m, 2H, m-CH_{Ar}).

¹³C NMR (100 MHz, DMSO-d₆): δ=153.6 (C═O), 133.9 (o-CH_{Ar}), 132.0 (m-CH_{Ar}), 123.7 (p-Br—C_{Ar}), 119.1 (i-C_{Ar}), 85.1 (C≡C), 81.6 (C≡C).

MS (EI, 70 eV): m/z (%)=225/223 (64/65) [M]⁺, 209/207 (95/100) [M-NH₂]⁺, 128 (21) [M-NH₂—Br]⁺, 99 (15), 75 (29), 74 (50), no other peaks >10%.

Anal. Calcd for C₉H₆BrNO: C, 48.25; H, 2.70; Br, 35.66; N, 6.25. Found: C, 48.04; H, 2.83; N, 6.24.

### 3-(4-Nitrophenyl)prop-2-ynamide (1e)

Colorless crystals; yield: 1.91 g (67%); mp 192-194° C. (dec) (MeCN).

IR (KBr): 3406 and 3155 (NH₂), 2220 (C≡C), 1662 cm⁻¹ (C═O).

¹H NMR (400 MHz, DMSO-d₆): δ=8.34 (s, 1H, NH₂), 7.88 (s, 1H, NH₂), 7.30 (m, 2H, o-CH_{Ar}), 7.85 (m, 2H, m-CH_{Ar}).

¹³C NMR (100 MHz, DMSO-d₆): δ=153.2 (C═O), 147.8 (p-NO₂—C_{Ar}), 133.3 (o-CH_{Ar}), 126.6 (i-C_{Ar}), 124.0 (m-CH_{Ar}), 87.8 (C≡C), 80.6 (C≡C).

MS (EI, 70 eV): m/z (%)=190 (84) [M]⁺, 174 (100) [M-NH₂]⁺, 128 (70) [M-NH₂—NO₂]⁺, 116 (26), 101 (16), 100 (28), 98 (13), 89 (50), 77 (15), 75 (30), 74 (54), 63 (19), 62 (20), 51 (23), 50 (23), no other peaks >10%.

Anal. Calcd for C₉H₆N₂O₃: C, 56.85; H, 3.18; N, 14.73. Found: C, 56.64; H, 3.24; N, 14.59.

### Monosubstituted Malonyl Chlorides (6a-f); General Procedure

Monosubstituted malonyl chlorides (6a-f) were obtained by refluxing the corresponding malonic acid with SOCl₂ and subsequent distillation. In the case of phenyl-6e and benzylmalonyl chloride 6f the corresponding (chlorocarbonyl)ethylketenes 7a,b were formed as a byproduct, which is in accordance with literature reports. (Nakanishi et al. Org Prep Proced. Int. (1975) 7:155; Friedrichsen et al. Naturforsch. B. Chem Sci (1982) 37:222; Chem. Abstr (1982) 96:199624).

### 2-(Phenylethynyl)-6H-1,3-oxazin-6-ones (8a-f); General Procedure

The corresponding malonyl chloride [(chlorocarbonyl)ethylketene] 6a-f (7a,b) (3.1 mmol) was added to a stirred soln of 3-phenylprop-2-ynamide (1a, 0.44 g, 3 mmol) in anhyd Et₂O (30 mL) at r.t. The mixture was cooled in the refrigerator for 12-14 h. During this time a solid deposited that was filtered off, washed with Et₂O, and dried to deliver pure 8a-f.

For an additional portion of product the filtrate was evaporated under reduced pressure at r.t. and the remaining residue was recrystallized (MeCN).

### 4-Hydroxy-5-methyl-2-(phenylethynyl)-6H-1,3-oxazin-6-one (8a) (Also Referred to as Compound PG-3a)

Yellowish crystals; yield: 0.32 g (74%); mp 184-186° C.

IR (KBr): 2215 (C≡C), 1747 (C═O), 1635 cm⁻¹ (C═N).

¹H NMR (400 MHz, DMSO-d₆): δ=12.74 (br s, 1H, OH), 7.71 (m, 2H, o-CH_{Ar}), 7.61 (m, 1H, p-CH_{Ar}), 7.52 (m, 2H, m-CH_{Ar}), 1.80 (s, 3H, CH₃).

¹³C NMR (100 MHz, DMSO-d₆): δ=164.8 (C6), 161.3 (C4), 146.9 (C2), 132.7 (o-CH_{Ar}), 131.5 (p-CH_{Ar}), 129.2 (m-CH_{Ar}), 118.4 (i-C_{Ar}), 92.8 (C5), 91.2 (C2′), 80.7 (C1′), 8.1 (CH₃).

MS (EI, 70 eV): m/z (%)=227 (14) [M]⁺, 171 (20), 130 (11), 129 (100) [PhC≡CCO]⁺, 128 (12), 83 (14), 77 (13) [Ph]⁺, 75 (29), 74 (14), 70 (12), 63 (11), no other peaks >10%.

Anal. Calcd for C₁₃H₉NO₃: C, 68.72; H, 3.99; N, 6.16. Found: C, 68.72; H, 4.01; N, 6.26.

### 5-Ethyl-4-hydroxy-2-(phenylethynyl)-6H-1,3-oxazin-6-one (8b) (Also Referred to as Compound PG-3b)

Yellowish crystals; yield: 0.51 g (70%); mp 177-178° C.

IR (KBr): 2221 (C≡C), 1744 (C═O), 1633 cm⁻¹ (C═N).

¹H NMR (400 MHz, DMSO-d₆): δ=12.77 (br s, 1H, OH), 7.71 (m, 2H, o-CH_{Ar}), 7.61 (m, 1H, p-CH_{Ar}), 7.53 (m, 2H, m-CH_{Ar}), 2.29 (q, ³J_{HH}=7.42 Hz, 2H, CH₂), 1.01 (t, ³J_{HH}=7.42 Hz, 3H, CH₃).

¹³C NMR (100 MHz, DMSO-d₆): δ=164.6 (C6), 160.8 (C4), 147.2 (C2), 132.7 (o-CH_{Ar}), 131.7 (p-CH_{Ar}), 129.1 (m-CH_{Ar}), 118.4 (i-C_{Ar}), 98.6 (C5), 91.1 (C2′), 80.6 (C1′), 16.1 (CH₂), 12.0 (CH₃).

MS (EI, 70 eV): m/z (%)=241 (28) [M]⁺, 129 (100) [PhC≡CCO]⁺, 128 (19), 75 (15), 51 (10), no other peaks >10%.

Anal. Calcd for C₁₄H₁₁NO₃: C, 69.70; H, 4.60; N, 5.81. Found: C, 69.42; H, 4.53; N, 5.84.

### 4-Hydroxy-5-isopropyl-2-(phenylethynyl)-6H-1,3-oxazin-6-one (8c) (Also Referred to as Compound PG-3c)

Colorless crystals; yield: 0.47 g (62%); mp 202-203° C.

IR (KBr): 2226 (C≡C), 1744 (C═O), 1627 cm⁻¹ (C═N).

¹H NMR (400 MHz, DMSO-d₆): δ=12.76 (br s, 1H, OH), 7.70 (m, 2H, o-CH_{Ar}), 7.61 (m, 1H, p-CH_{Ar}), 7.52 (m, 2H, m-CH_{Ar}), 3.01 (sept, ³J_{HH}=7.00 Hz, 1H, CH), 1.17 (d, ³J_{HH}=7.00 Hz, 6H, CH₃).

¹³C NMR (100 MHz, DMSO-d₆): δ=164.4 (C6), 160.0 (C4), 147.4 (C2), 132.8 (o-CH_{Ar}), 131.6 (p-CH_{Ar}), 129.2 (m-CH_{Ar}), 118.5 (i-C_{Ar}), 101.9 (C5), 91.2 (C2′), 80.6 (C1′), 23.7 (>CH—), 19.5 (CH₃).

MS (EI, 70 eV): m/z (%)=255 (35) [M]⁺, 240 (54) [M-CH₃]⁺, 172 (11), 130 (10), 129 (100) [PhC≡CCO]⁺, 128 (20), 75 (11), 69 (16), no other peaks >10%.

Anal. Calcd for C₁₅H₁₃NO₃: C, 70.58; H, 5.13; N, 5.49. Found: C, 70.38; H, 5.32; N, 5.43.

### 5-Butyl-4-hydroxy-2-(phenylethynyl)-6H-1,3-oxazin-6-one (8d) (Also Referred to as Compound PG-3)

Yellow crystals; yield: 0.53 g (65%); mp 157-159° C.

IR (KBr): 2222 (C≡C), 1743 (C═O), 1628 cm⁻¹ (C═N).

¹H NMR (400 MHz, DMSO-d₆): δ=12.71 (br s, 1H, OH), 7.70 (m, 2H, o-CH_{Ar}), 7.61 (m, 1H, p-CH_{Ar}), 7.52 (m, 2H, m-CH_{Ar}), 2.28 (m, 2H, CH₂), 1.42 (m, 2H, CH₂), 1.29 (m, 2H, CH₂), 0.88 (m, 3H, CH₃).

¹³C NMR (100 MHz, DMSO-d₆): δ=164.9 (C6), 161.0 (C4), 147.2 (C2), 132.7 (o-CH_{Ar}), 131.5 (p-CH_{Ar}), 129.2 (m-CH_{Ar}), 118.4 (i-C_{Ar}), 97.3 (C5), 91.2 (C2′), 80.7 (C1′), 29.3 (C2″), 22.3 (C1″), 21.9 (C3″), 13.7 (C4″).

MS (EI, 70 eV): m/z (%)=269 (10) [M]⁺, 226 (18) [M-C₃H₇]⁺, 165 (14), 130 (10), 129 (100) [PhC≡CCO]⁺, 128 (20), no other peaks >10%.

Anal. Calcd for C₁₆H₁₅NO₃: C, 71.36; H, 5.61; N, 5.20. Found: C, 70.97; H, 5.46; N, 5.14.

### 4-Hydroxy-5-phenyl-2-(phenylethynyl)-6H-1,3-oxazin-6-one (8e) (Komarov et al. Russ. J. Gen Chem (2005) 75:770) (Also Referred to as Compound PG-3e)

Yellow crystals; yield: 0.76 g (87%); mp 187-189° C.

IR (KBr): 2220 (C≡C), 1747 (C═O), 1620 cm⁻¹ (C═N).

¹H NMR (400 MHz, DMSO-d₆): δ=13.18 (br s, 1H, OH), 7.74 (m, 2H, o-CH_{Ar}), 7.63 (m, 1H, p-CH_{Ar}), 7.54 (m, 2H, m-CH_{Ar}), 7.52 (m, 2H, H2″), 7.38 (m, 2H, H3″), 7.29 (m, 1H, H4″).

¹³C NMR (100 MHz, DMSO-d₆): δ=164.8 (C6), 160.0 (C4), 147.9 (C2), 132.8 (o-CH_{Ar}), 131.7 (p-CH_{Ar}), 130.6 (C1″), 130.0 (C2″), 129.2 (m-CH_{Ar}), 127.6 (C3″), 127.2 (C4″), 118.3 (i-C_{Ar}), 97.3 (C5), 91.8 (C2′), 80.8 (C1′).

MS (EI, 70 eV): m/z (%)=289 (35) [M]⁺, 218 (14), 190 (12), 172 (12), 130 (10), 129 (100) [PhC≡CCO]⁺, 118 (18), 89 (16), 77 (11) [Ph]⁺, 75 (13), 51 (10) [C₄H₃]⁺, no other peaks >10%.

Anal. Calcd for C₁₈H₁₁NO₃: C, 74.73; H, 3.83; N, 4.84. Found: C, 74.79; H, 4.01; N, 4.75.

### 5-Benzyl-4-hydroxy-2-(phenylethynyl)-6H-1,3-oxazin-6-one (8f) (Also Referred to as PG-3d)

Yellow crystals; yield: 0.55 g (61%); mp 179-181° C.

IR (KBr): 2221 (C≡C), 1746 (C═O), 1627 cm⁻¹ (C═N).

¹H NMR (400 MHz, DMSO-d₆): δ=13.03 (br s, 1H, OH), 7.71 (m, 2H, o-CH_{Ar}), 7.60 (m, 1H, p-CH_{Ar}), 7.52 (m, 2H, m-CH_{Ar}), 7.25 (m, 4H, H2″, H3″), 7.17 (m, 1H, H4″), 3.61 (s, 2H, CH₂).

¹³C NMR (100 MHz, DMSO-d₆): δ=165.4 (C6), 161.0 (C4), 147.7 (C2), 139.3 (C1″), 132.7 (o-CH_{Ar}), 131.6 (p-CH_{Ar}), 129.2 (m-CH_{Ar}), 128.21 and 128.18 (C2″ and C3″), 126.0 (C4″), 118.4 (i-C_{Ar}), 96.5 (C5), 91.5 (C2′), 80.7 (C1′), 28.3 (CH₂).

MS (EI, 70 eV): m/z (%)=302 (55) [M]⁺, 176 (28), 158 (74), 131 (15), 130 (65), 129 (100) [PhC≡CCO]⁺, 128 (20), 103 (12), 102 (15), 91 (27) [PhCH₂]⁺, 77 (22) [Ph]⁺, 75 (13), 51 (12) [C₄H₃]⁺, no other peaks >10%.

Anal. Calcd for C₁₉H₁₃NO₃: C, 75.24; H, 4.32; N, 4.62. Found: C, 75.27; H, 4.32; N, 4.65.

### 2-[(4-Chlorophenyl)ethynyl]-4-hydroxy-5-phenyl-6H-1,3-oxazin-6-one (Also Referred to as PG-3g)

Yellow crystals; yield: 0.87 g (90%); mp 185-187° C. (dec).

IR (KBr): 2220 (C≡C), 1767 (C═O), 1620 cm⁻¹ (C═N).

¹H NMR (400 MHz, DMSO-d₆): δ=13.16 (br s, 1H, OH), 7.77 (m, 2H, o-CH_{Ar}), 7.62 (m, 2H, m-CH_{Ar}), 7.52 (m, 2H, H2″), 7.37 (m, 2H, H3″), 7.23 (m, 1H, H4″).

¹³C NMR (100 MHz, DMSO-d₆): δ=164.7 (C6), 159.9 (C4), 147.6 (C2), 136.6 (p-ClC_{Ar}), 134.5 (o-CH_{Ar}), 130.5 (C1″), 130.0 (C2″), 129.4 (m-CH_{Ar}), 127.5 (C3″), 127.2 (C4″), 117.2 (i-C_{Ar}), 97.4 (C5), 90.3 (C2′), 81.5 (C1′).

MS (EI, 70 eV): m/z (%)=325/324/323 (15/9/47) [M]⁺, 254/252 (9/22), 225 (10), 224 (22), 206 (16), 165/163 (32/100) [4-Cl—C₆H₄C≡CCO]⁺, 118 (28), 99 (18), 90 (17), 89 (21), 77 (12) [Ph]⁺, no other peaks >10%.

Anal. Calcd for C18H10ClNO3: C, 66.78; H, 3.11; N, 4.33. Found: C, 66.89; H, 3.10; N, 4.39.

### 2-[(2-Chlorophenyl)ethynyl]-4-hydroxy-5-methyl-6H-1,3-oxazin-6-one (Also Referred to as PG-3f)

Yellowish crystals; yield: 0.46 g (59%); mp 175-178° C.

IR (KBr): 2225 (C≡C), 1747 (C═O), 1627 cm⁻¹ (C═N).

¹H NMR (400 MHz, DMSO-d₆): δ=12.83 (br s, 1H, OH), 7.83 (m, 1H, H6′), 7.69 (m, 1H, H3′), 7.62 (m, 1H, H4′), 7.50 (m, 1H, H5′), 1.80 (s, 3H, CH₃).

¹³C NMR (100 MHz, DMSO-d₆): δ=165.0 (C6), 161.3 (C4), 146.6 (C2), 135.8 (C2′), 134.9 (C6′), 133.1 (C4′), 129.9 (C3′), 127.8 (C5′), 118.6 (C1′), 92.8 (C5), 86.9 (C2′), 84.9 (C1′), 8.3 (CH₃).

MS (EI, 70 eV): m/z (%)=263/262/261 (9/4/26) [M]⁺.

Anal. Calcd for C13H8ClNO3: C, 59.67; H, 3.08; N, 5.35.

### 3-Oxo-3-[(3-phenylprop-2-ynoyl)amino]propanoic Acids (9a-f); General Procedure

After keeping a soln of compounds 8a-f in DMSO-d₆ for 24 h at r.t. the ¹H and ¹³C NMR spectra of the 4-hydroxy-2-(phenylethynyl)-6H-1,3-oxazin-6-ones 8a-f showed new signals of the corresponding hydrolysis products 9a-d,f and 10 (as shown in FIG. 2).

### 2-Methyl-3-oxo-3-[(3-phenylprop-2-ynoyl)amino]propanoic Acid (9a)

¹H NMR (400 MHz, DMSO-d₆): δ=12.79 (br s, 1H, OH), 11.62 (s, 1H, NH), 7.66 (m, 2H, o-CH_{Ar}), 7.58 (m, 1H, p-CH_{Ar}), 7.50 (m, 2H, m-CH_{Ar}), 3.79 (q, ³J_{HH}=7.15 Hz, 1H, H2), 1.27 (t, ³J_{HH}=7.15 Hz, 3H, CH₃).

¹³C NMR (100 MHz, DMSO-d₆): δ=171.3 (C1), 170.1 (C3), 151.5 (C5), 132.6 (o-CH_{Ar}), 131.1 (p-CH_{Ar}), 129.0 (m-CH_{Ar}), 119.0 (i-C_{Ar}), 88.9 (C7), 83.2 (C6), 47.2 (C2), 13.1 (CH₃).

### 2-[(3-Phenylprop-2-ynoyl)carbamoyl]butanoic Acid (9b)

¹H NMR (400 MHz, DMSO-d₆): δ=12.84 (br s, 1H, OH), 11.65 (s, 1H, NH), 7.66 (m, 2H, o-CH_{Ar}), 7.58 (m, 1H, p-CH_{Ar}), 7.50 (m, 2H, m-CH_{Ar}), 3.61 (m, 1H, H2), 1.80 (m, 2H, CH₂), 0.90 (m, 3H, CH₃).

¹³C NMR (100 MHz, DMSO-d₆): δ=170.3 (C1), 169.2 (C3), 151.5 (C5), 132.7 (o-CH_{Ar}), 131.2 (p-CH_{Ar}), 129.1 (m-CH_{Ar}), 119.0 (i-C_{Ar}), 89.0 (C7), 83.1 (C6), 54.2 (C2), 21.4 (CH₂), 11.8 (CH₃).

### 3-Methyl-2-[(3-phenylprop-2-ynoyl)carbamoyl]butanoic Acid (9c)

¹H NMR (400 MHz, DMSO-d₆): δ=12.74 (br s, 1H, OH), 11.60 (s, 1H, NH), 7.66 (m, 2H, o-CH_{Ar}), 7.58 (m, 1H, p-CH_{Ar}), 7.51 (m, 2H, m-CH_{Ar}), 3.46 (m, 1H, H2), 2.28 (m, 1H, CH), 0.97 (m, 3H, CH₃).

¹³C NMR (100 MHz, DMSO-d₆): δ=169.6 (C1), 168.4 (C3), 151.4 (C5), 132.6 (o-CH_{Ar}), 131.2 (p-CH_{Ar}), 129.0 (m-CH_{Ar}), 119.0 (i-C_{Ar}), 89.1 (C7), 83.1 (C6), 59.3 (C2), 28.0 (CH), 20.2 (CH₃), 20.0 (CH₃).

### 2-[(3-Phenylprop-2-ynoyl)carbamoyl]hexanoic Acid (9d)

¹H NMR (400 MHz, DMSO-d₆): δ=12.82 (br s, 1H, OH), 11.62 (s, 1H, NH), 7.66 (m, 2H, o-CH_{Ar}), 7.58 (m, 1H, p-CH_{Ar}), 7.50 (m, 2H, m-CH_{Ar}), 3.67 (m, 1H, H2), 1.78 (m, 2H, H1′), 1.28 (m, 4H, H2′, H3′), 0.87 (m, 3H, H4).

¹³C NMR (100 MHz, DMSO-d₆): δ=170.4 (C1), 169.2 (C3), 151.5 (C5), 132.6 (o-CH_{Ar}), 131.2 (p-CH_{Ar}), 129.0 (m-CH_{Ar}), 119.0 (i-C_{Ar}), 89.0 (C7), 83.1 (C6), 52.7 (C2), 29.1 (C3′), 27.7 (C1′), 21.9 (C2′), 13.7 (C4′).

### 2-Benzyl-3-oxo-3-[(3-phenylprop-2-ynoyl)amino]propanoic Acid (9f)

¹H NMR (400 MHz, DMSO-d₆): δ=13.03 (br s, 1H, OH), 11.62 (s, 1H, NH), 7.65 (m, 2H, o-CH_{Ar}), 7.59 (m, 1H, p-CH_{Ar}), 7.52 (m, 2H, m-CH_{Ar}), 7.25 (m, 5H, H2′, H3′, H4′), 4.07 (t, ³J_{HH}=7.46 Hz, 1H, H2), 3.12 (d, ³J_{HH}=7.46 Hz, 2H, CH₂).

¹³C NMR (100 MHz, DMSO-d₆): δ=169.7 (C1), 168.5 (C3), 151.4 (C5), 138.4 (C1′), 132.6 (o-CH_{Ar}), 131.2 (p-CH_{Ar}), 129.0 (m-CH_{Ar}), 128.7 (C2′), 128.3 (C3′), 126.4 (C4′), 118.9 (i-C_{Ar}), 89.1 (C7), 83.0 (C6), 54.4 (C2), 33.6 (CH₂).

### 3-Phenyl-N-(phenylacetyl)prop-2-ynamide (10) (Also Referred to as Compound PG-3h)

A suspension of 4-hydroxy-5-phenyl-2-(phenylethynyl)-6H-1,3-oxazin-6-one (8e, 0.3 mmol) in H₂O (20 mL) was heated under reflux for 30 min until the color of 8e turned from yellow to white. The solid compound 10 was filtered off and recrystallized (toluene) to give colorless crystals; yield: 71 mg (90%); mp 132-134° C.

IR (KBr): 3219 (NH), 2213 (C≡C), 1705 (C═O), 1683 cm⁻¹ (C═O).

¹H NMR (400 MHz, DMSO-d₆): δ=11.60 (s, 1H, NH), 7.64 (m, 2H, o-CH_{Ar}), 7.56 (m, 1H, p-CH_{Ar}), 7.49 (m, 2H, m-CH_{Ar}), 7.33 (m, 2H, H3′), 7.27 (m, 3H, H2′, H4′), 3.82 (s, 2H, CH₂).

¹³C NMR (100 MHz, DMSO-d₆): δ=170.7 (C═O), 151.6 (C1), 134.3 (C1′), 132.6 (o-CH_{Ar}), 131.1 (p-CH_{Ar}), 129.5 (C3′), 129.0 (m-CH_{Ar}), 128.3 (C2′), 126.8 (C4′), 119.1 (i-C_{Ar}), 89.0 (C3), 83.3 (C2), 43.1 (CH₂).

MS (EI, 70 eV): m/z (%)=263 (26) [M]⁺, 147 (18), 251 (15), 130 (10), 129 (94) [PhC≡CCO]⁺, 118 (100), 117 (12), 105 (15), 91 (30) [PhCH₂]⁺, 90 (22), 75 (13), 65 (11), no other peaks >10%.

Anal. Calcd for C₁₇H₁₃NO₂: C, 77.55; H, 4.98; N, 5.32. Found: C, 77.49; H, 5.07; N, 5.25.

### EXAMPLE 1

### PG3 Compounds Inhibit Caspase-6 as Measured by the FRET Assay

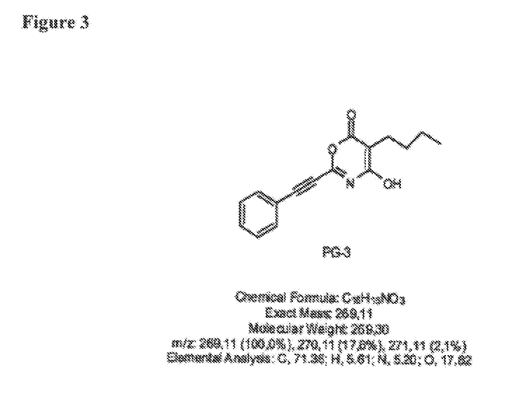

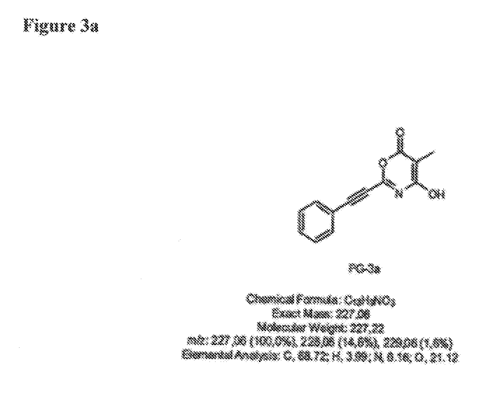

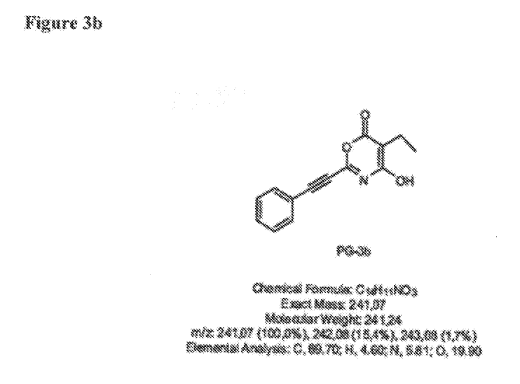

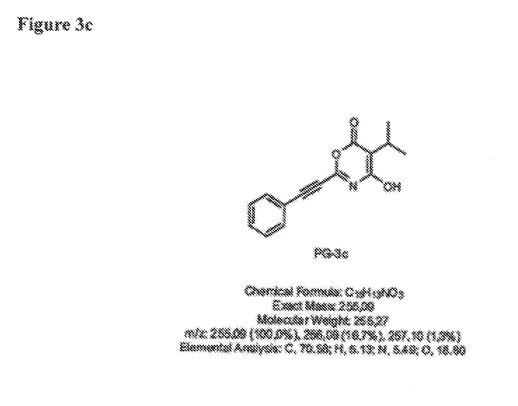

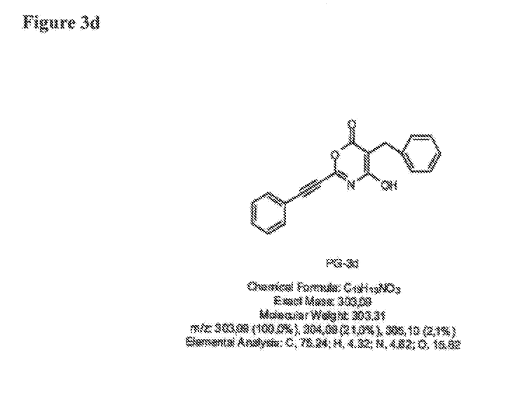

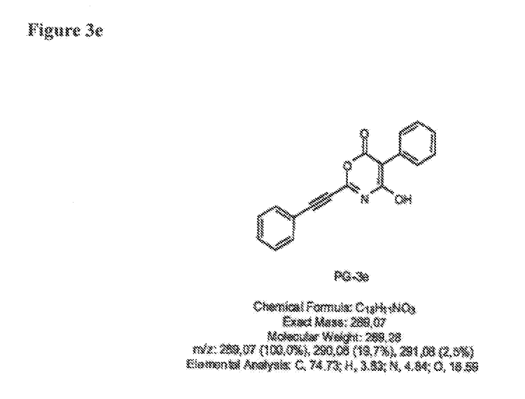

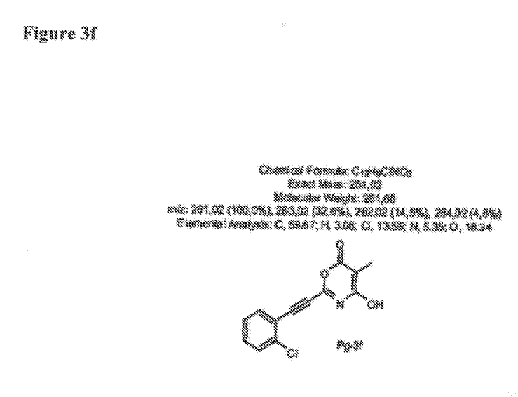

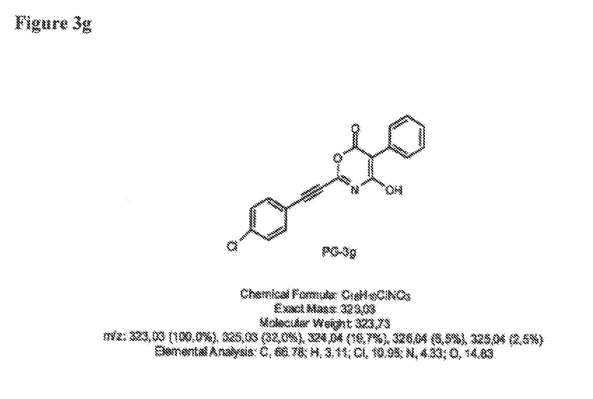

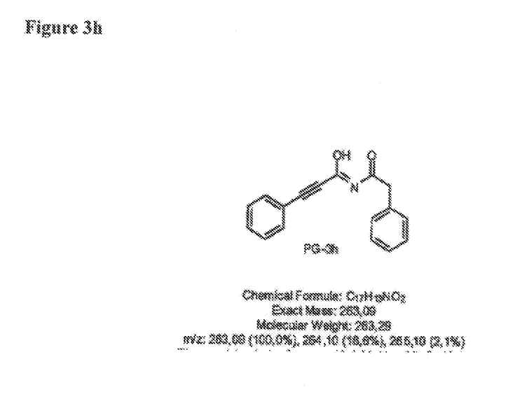

FIG. 3 shows the compounds (referred to as PG3a-h) that were tested in dose-response curves with the FRET assay. As shown in FIG. 4A-C, the presence of increasing concentrations of several of the PG3 analogs resulted in increased inhibition of the recombinant caspase-6 enzyme in a cell-free system. Table 3 shows the calculated IC₅₀ values for the inhibition of the caspase-6 enzyme as measured by the FRET assay.

TABLE 3
The PG3 compound and its analogs
(PG-3a-h) have inhibitory effects on
recombinant caspase-6

| Compound | FRET IC₅₀ [μM] |
|---|---|
| PG-3 | 13.8 |
| PG-3a | 8.2 |
| PG-3b | 8.0 |
| PG-3c | 12.2 |
| PG-3d | 5.6 |
| PG-3e | 3.9 |
| PG-3f | 57.3 |
| PG-3g | 7.5 |
| PG-3h | 8.1 |

### EXAMPLE 2

### PG3 Compounds Inhibit Intracellular Caspase-6 as Measured by Western Blot

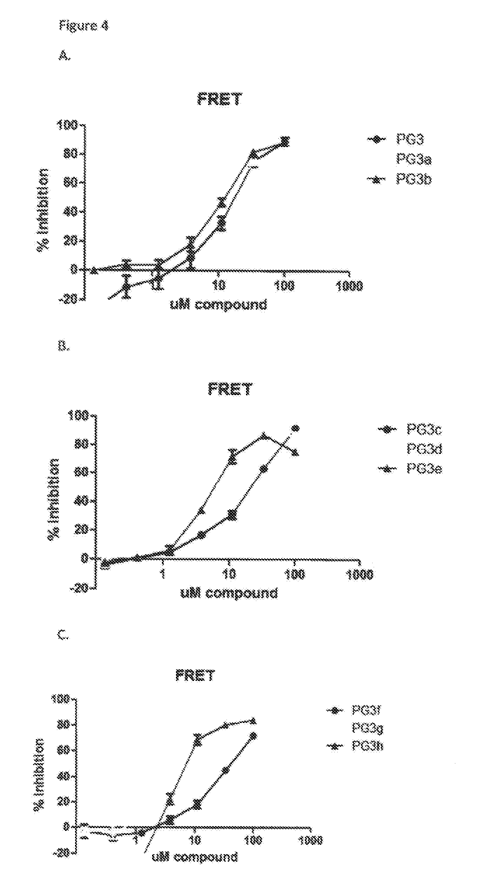

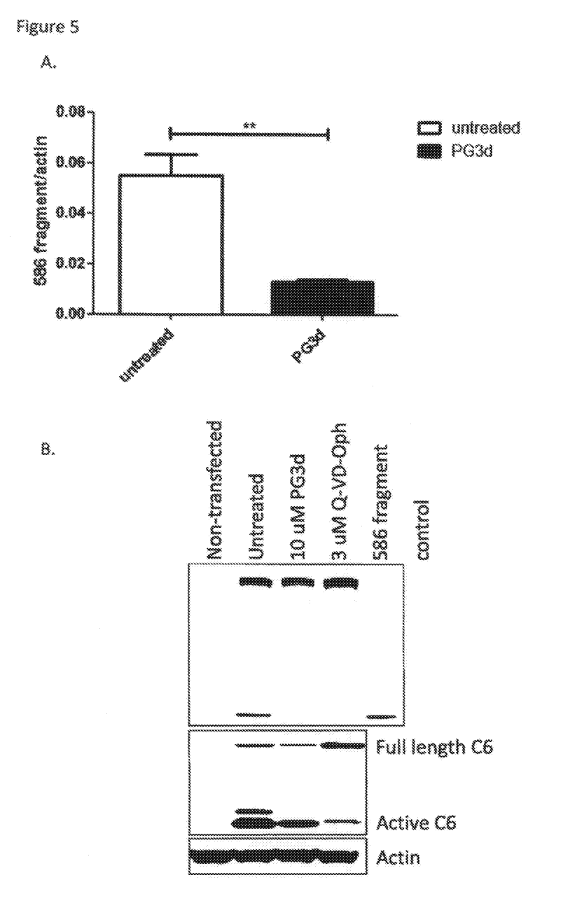

COS-7 cells were co-transfected with the 4c htt fragment and the human caspase-6 lacking the pro-domain which leads to fast autoactivation of the caspase-6 enzyme and the generation of the 586 aa Htt cleavage fragment. As shown in FIG. 5B, the co-transfected cells were exposed to either 10 um of the PG-3d compound, 3 uM of the Q-VD-Oph pan-caspase inhibitor or were left untreated. Non-transfected cells were included as a negative control and the purified 586 aa Htt fragment was included as a positive control. Cells that were exposed to the PG-3d compound show a significant reduction in the generation of the 586AA fragment. The cell lysates were also assayed for the levels of active caspase 6 enzyme as shown in the lower panel of FIG. 5B. The presence of the PG-3d compound resulted in a decreased amount of active enzyme which was also achieved in the presence of pan-caspase inhibitor. FIG. 5A shows a graphical representation from the Western blot, and indicated that the presence of PG-3d resulted in a reduced production of the 586 aa fragment.

### EXAMPLE 3

### PG3 Compounds Inhibit Intracellular Caspase-6 as Measured by Lamin Cleavage

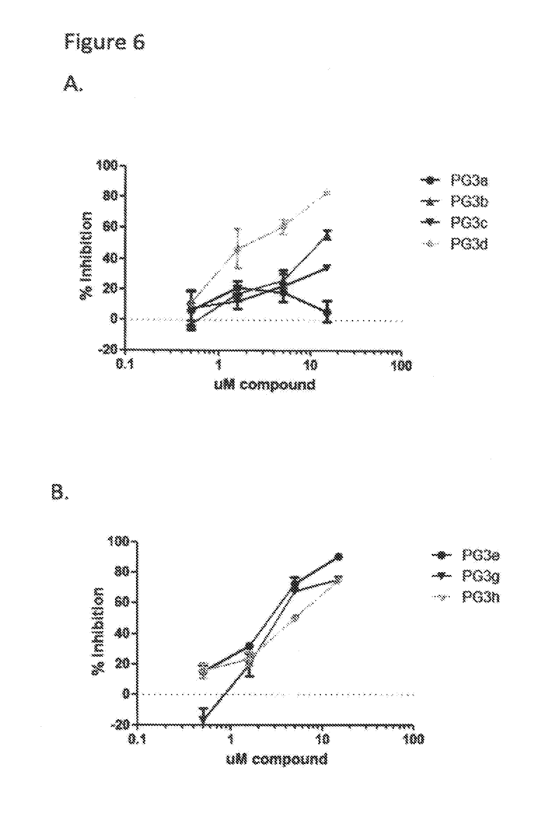

The PG3 analog compounds were also tested for their ability to inhibit caspase-6 within cultured cells. HEK 293 cells were transfected with human caspase 6 lacking the pro-domain and were incubated with increasing concentrations of the PG3 analogs. After a 24 hr incubation, the excess compound was washed away, the cells were lysed and the amount of cleaved lamin A protein was quantified by the Mesoscale ELISA method. As shown in FIGS. 6A and 6B, several of the PG3 compounds demonstrated a dose-dependent ability to inhibit the caspase-6-mediated cleavage of lamin. Table 4 shows the calculated IC₅₀ values for the intracellular inhibition of caspase-6 for the PG3 analogs.

TABLE 4
The PG3 analogs have an inhibitory
effect on caspase-6 within cultured cells

| Compound | HEK intracellular IC₅₀ [μM] |
|---|---|
| PG-3 | n.a. |
| PG-3a | >15 |
| PG-3b | 12.1 |
| PG-3c | >15 |
| PG-3d | 2.6 |
| PG-3e | 2.5 |
| PG-3f | n.a. |
| PG-3g | 3.9 |
| PG-3h | 4.8 |

### EXAMPLE 4

### PG3 Compounds Inhibit Neuronal Caspase-6 as Measured by Lamin Cleavage

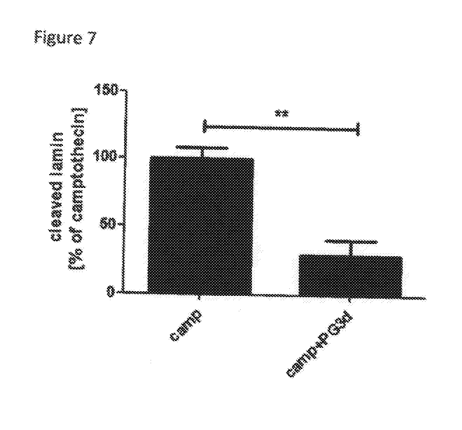

Next, it was determined whether the PG-3d compound could inhibit caspase-6 within neuronal cells in an in vitro neuronal system which may be more relevant for the testing of therapeutics for neurodegeneration. Briefly, primary cortical neuronal cultures from FVB/N mice were treated with 10 uM camptothecin for 30 h starting at DIV10 in the presence or absence of 10 uM of the PG-3d compound. Camptothecin treatment leads to the activation of caspase-6 which was then quantified by the cleavage of lamin A. As shown in FIG. 7, the presence of the PG3d compound in the neuronal medium significantly reduces the level of caspase-6 activity as evidenced by the reduction in cleaved lamin.

### EXAMPLE 5

### PG3 Compounds Improve the Viability of Neuronal Cells During Excitotoxic Stress

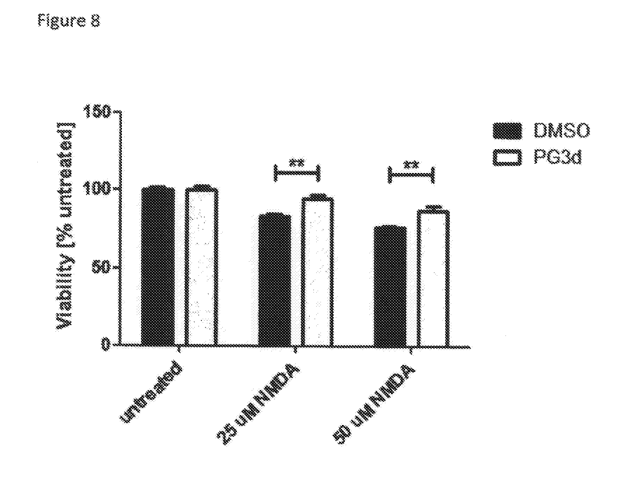

It has been previously been shown in HD animal models that neuronal cells expressing mHTT have an increased vulnerability to excitotoxic stress (Graham et al. 2005. Neurobiol. Dis. 21:444-455) and that this may be mediated by caspase 6 (Graham et al. 2006. Cell 6(13):1179-1191 and Uribe et al HMG 2012; 21(9):1954-67). The link between NMDA-induced toxicity and other neurodegenerative diseases has been reviewed by Lipton et al., Nat Rev Drug Discovery 2006. 5:160-170. Therefore, the effect of the presence of the PG-3d compound on the ability of neuronal cells to survive during excitotoxic stress via its ability to inhibit the caspase-6 enzyme was investigated. As shown in FIG. 8, the presence of the PG-3d compound in the cell culture medium resulted in a significant improvement in cell viability in neuronal cells that were exposed to NMDA (25 uM or 50 uM).

### EXAMPLE 6

### PG3d Inhibits the Interaction Between Caspase-6 and Htt in Mammalian Cells

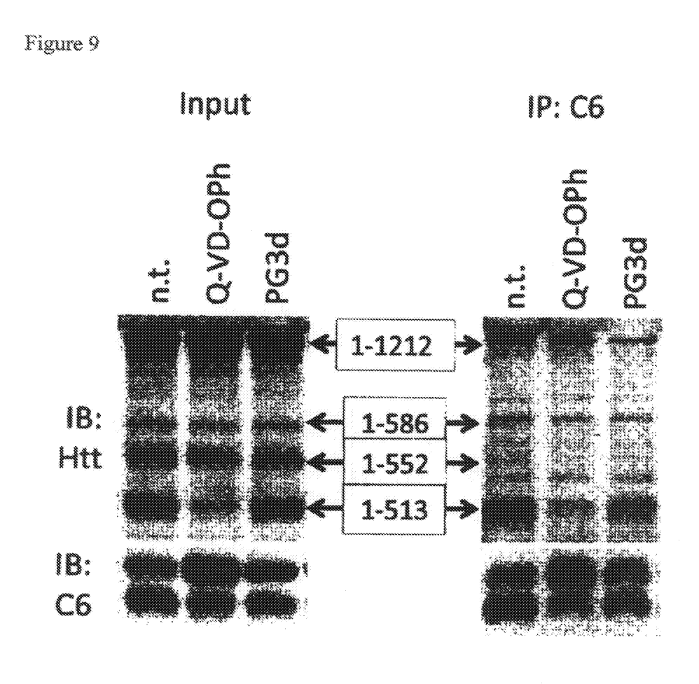

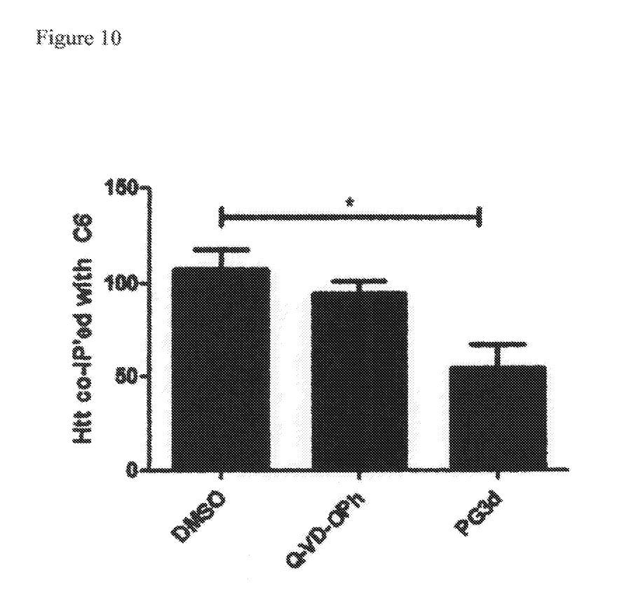

Cos-7 cells were co-transfected with Htt 1-1212AA (SEQ ID NO:3) and full-length human caspase-6 (SEQ ID NO:6), and exposed to either the pan-caspase inhibitor Q-VD-OPh, the PG3d compound or DMSO as a negative control. As shown in the upper panel of FIG. 9A, control cells that received DMSO alone (lanes labelled n.t.) show the presence of the Htt 1212AA protein and the Htt proteolytic cleavage fragments including the 586, 552 and 513 amino acid fragments. As expected, cells treated with the pan-caspase inhibitor Q-VD-OPh show a reduction in the amount of Htt cleavage fragments most markedly in the 513AA fragment derived from Caspase 3 activity. Cells treated with the PG3d compound show a reduction in the level of the 586AA fragment derived from caspase 6 activity (FIG. 9A, upper panel).

FIG. 9B shows the results following immunoprecipitaton with a caspase-6 antibody. As expected, the DMSO control lysates reveal a variety of Htt fragments within the immunocomplexes, including 513AA fragment generated by caspase-3 cleavage, the full-length 1212AA Htt and the 586AA fragment generated by caspase-6 cleavage. When the cells are treated with the pan-caspase inhibitor Q-VD-OPh, the amount of Htt cleavage fragments interacting with caspase-6 is reduced and it is evident that caspase-6 is primarily interacting with the 1212 AA Htt. Treatment with 10 uM PG3d significantly reduces the interaction between the 1212AA Htt and caspase-6 which also results in a reduced amount of co-immunoprecipitated Htt proteolytic fragments. These findings were analyzed by densitometric analysis of the immunoblot results in FIG. 9B and confirm that that presence of PG3d leads to a significant reduction in the amount of Htt 1212 AA that is co-immunoprecipitated using a Caspase-6 antibody as compared to the negative control DMSO-treated cells or cells treated with the Q-VD-OPh peptide inhibitor.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

1. A pharmaceutical composition for the treatment or amelioration of a neurological disease wherein the composition comprises a therapeutically effective amount of an active agent with caspase-6 inhibitory activity and one or more pharmaceutically acceptable excipients, wherein the active agent has one of the following structures I or II, or a pharmaceutically acceptable salt, stereoscopic isomer, derivative or prodrug thereof: 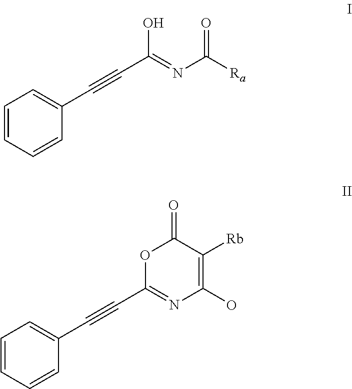
wherein Ra and Rb are independently linear or branched C₁ to C₆ alkyl, aryl, or alkenyl, C₁ to C₉ alkylaryl, C₁ to C₉ substituted alkylaryl, or C₁ to C₉ alkylheteroaryl; and
wherein the phenyl group is substituted with 0, 1 or 2 halogens and further wherein the halogens are chloride, fluoride or bromide.

2. The pharmaceutical composition of claim 1, wherein the composition is formulated for oral or topical administration, subcutaneous, intravenous, or intramuscular injection, infusion, inhalation, or intrathecal injection directly into the central nervous system.

3. The pharmaceutical composition of claim 1, wherein the neurological disease is Huntington's disease, Alzheimer's disease, dementia, mild-cognitive impairment, or memory loss.

4. A method of treating a neurological disease comprising administering to a subject in need of such treatment an effective dose of a pharmaceutical composition of claim 1 wherein said disease is Huntington's disease, Alzheimer's disease, dementia, mild-cognitive impairment, or memory loss.

5. The method of claim 4 wherein the composition of claim 1 is administered in combination with one or more additional drugs useful in the treatment of neurological disease.

6. The method of claim 5 wherein the one or more additional drugs is selected from L-DOPA, rasagiline, memantine hydrochloride, donepezil hydrochloride, rivastigmine, galantamine and tetrabenzine.

7. The method of claim 4, wherein administration of an effective dose of a pharmaceutical composition of claim 1 is commenced prior to the appearance of symptoms of said neurological disease in said subject.

8. The method of claim 7, wherein cells of the subject express a mutant htt gene.

9. The method of claim 7, wherein neural cells of the subject overexpress caspase-6 mRNA.

10. A method according to claim 4 wherein the subject is human.

11. A pharmaceutical composition for the treatment or amelioration of a neurological disease wherein the composition comprises a therapeutically effective amount of an active agent with the structure designated as PG-3a in FIG. 3A, or pharmaceutically acceptable salt, stereoscopic isomer, derivative or prodrug thereof.

12. A pharmaceutical composition for the treatment or amelioration of a neurological disease wherein the composition comprises a therapeutically effective amount of an active agent with the structure designated as PG-3b in FIG. 3B, or pharmaceutically acceptable salt, stereoscopic isomer, derivative or prodrug thereof.

13. A pharmaceutical composition for the treatment or amelioration of a neurological disease wherein the composition comprises a therapeutically effective amount of an active agent with the structure designated as PG-3c in FIG. 3C, or pharmaceutically acceptable salt, stereoscopic isomer, derivative or prodrug thereof.

14. A pharmaceutical composition for the treatment or amelioration of a neurological disease wherein the composition comprises a therapeutically effective amount of an active agent with the structure designated as PG-3d in FIG. 3D, or pharmaceutically acceptable salt, stereoscopic isomer, derivative or prodrug thereof.

15. A pharmaceutical composition for the treatment or amelioration of a neurological disease wherein the composition comprises a therapeutically effective amount of an active agent with the structure designated as PG-3e in FIG. 3E, or pharmaceutically acceptable salt, stereoscopic isomer, derivative or prodrug thereof.

16. A pharmaceutical composition for the treatment or amelioration of a neurological disease wherein the composition comprises a therapeutically effective amount of an active agent with the structure designated as PG-3f in FIG. 3F, or pharmaceutically acceptable salt, stereoscopic isomer, derivative or prodrug thereof.

17. A pharmaceutical composition for the treatment or amelioration of a neurological disease wherein the composition comprises a therapeutically effective amount of an active agent with the structure designated as PG-3g in FIG. 3G, or pharmaceutically acceptable salt, stereoscopic isomer, derivative or prodrug thereof.

18. A pharmaceutical composition for the treatment or amelioration of a neurological disease wherein the composition comprises a therapeutically effective amount of an active agent with the structure designated as PG-3h in FIG. 3H, or pharmaceutically acceptable salt, stereoscopic isomer, derivative or prodrug thereof.

19. A pharmaceutical composition for the treatment or amelioration of a neurological disease wherein the composition comprises a therapeutically effective amount of an active agent with the structure designated as PG-3 in FIG. 3, or pharmaceutically acceptable salt, stereoscopic isomer, derivative or prodrug thereof.
